(19) United States
(12) Patent Application Publication
(10) Pub. No.: US 2026/0315538 A1
(43) Pub. Date: Oct. 15, 2026

(54) METHODS FOR RESTORING GUT MICROBIOME AFTER INTESTINAL SURGERY

(71) Applicant: REGENTS OF THE UNIVERSITY OF MINNESOTA, Minneapolis, MN (US)

(72) Inventors: Cyrus Jahansouz, Minneapolis, MN (US); Alexander Khoruts, Minneapolis, MN (US); Christopher M. Staley, Minneapolis, MN (US)

(21) Appl. No.: 19/576,017

(22) Filed: Mar. 24, 2026

Related U.S. Application Data
(60) Provisional application No. 63/777,822, filed on Mar. 26, 2025.

(51) Int. Cl. A61K 35/24; A61K 9/19; A61K 35/00; A61P 1/14

(52) U.S. Cl. CPC A61K 35/24; A61K 9/19; A61P 1/14; A61K 2035/115

(57) ABSTRACT

The present disclosure provides freeze-dried and liquid compositions that include an extract of human feces. Also provided are methods for making and using such compositions, including methods for replacing or supplementing or modifying a subject's intestinal microbiota, and methods for treating an individual that has undergone an intestinal surgery.

### CROSS-REFERENCE TO RELATED APPLICATIONS

This application claims the benefit of U.S. Provisional Application Ser. No. 63/777,822, filed Mar. 26, 2025, which is incorporated by reference herein in its entirety.

### SUMMARY OF THE APPLICATION

Provided herein are methods of using a fecal extract. In one embodiment, a method of the present disclosure includes decreasing the relative abundance of a member of the genus Streptococcus, a member of the genus Enterococcus, or a member of the genus Streptococcus and a member of the genus Enterococcus, in a patient that has undergone an intestinal surgery. The method includes administering to the patient an effective amount of a composition comprising a fecal extract and a pharmaceutically acceptable carrier, and results in the relative abundance of a member of the genus Streptococcus, a member of the genus Enterococcus, or a member of the genus Streptococcus and a member of the genus Enterococcus being reduced in the colon of the patient following administration of the composition.

In one embodiment, a method of the present disclosure includes increasing the relative abundance of one or more members of the family Bacteroidaceae, one or more members of the family Lachnospiraceae, one or more members of the family Ruminococcaceae, one or more members of the family Akkermansiaceae, one or more members of the family Coriobacteriaceae, or any combination thereof in a patient that has undergone an intestinal surgery. The method includes administering to the patient an effective amount of a composition comprising a fecal extract and a pharmaceutically acceptable carrier, and results in the relative abundance of one or more members of the family Bacteroidaceae, the family Lachnospiraceae, the family Ruminococcaceae, the family Akkermansiaceae, or the family Coriobacteriaceae being increased in the colon of the patient following administration of the composition.

In another embodiment, a method of the present disclosure includes increasing the presence of short-chain fatty acids (SCFAs), branched-chain fatty acids (BCFAs), or both SCFAs and BCFAs in a patient that has undergone an intestinal surgery. The method includes administering to the patient an effective amount of a composition comprising a fecal extract and a pharmaceutically acceptable carrier, and results in increasing in the colon of the patient the concentration of SCFAs, BCFAs, or both SCFAs and BCFAs following administration of the composition.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows Microbiota Transplant Therapy (MTT) promotes durable microbial engraftment. Engraftment was assessed as the percentage similarity between the fecal microbiota of transplanted patients and that of healthy donors using SourceTracker. Donor similarity increases by POD14 and remains elevated through 6 months. * denotes statistical significance at P<0.05; ** denotes statistical significance at P<0.005.

FIGS. 2A-2B show MTT alters microbial diversity and community structure after surgery. FIG. 2A, Relative abundance of predominant taxa in non-MTT controls and MTT recipients across perioperative timepoints, demonstrating postoperative collapse followed by divergent community recovery trajectories. Predominant taxa represent the most abundant genera across all samples, collapsed across timepoints for visualization. FIG. 2B, Principal coordinate analysis (PCoA) of Bray-Curtis distances showing fecal microbial community structure in control patients (all timepoints) and MTT recipients before (orange circles) and after (green circles) transplantation (r²=0.37). Samples from MTT recipients diverge away from both inpatient and baseline samples.

FIG. 3 shows alpha diversity between control and MTT groups. Shannon indices (alpha diversity) for non-MTT control and MTT patients at baseline, inpatient, POD14, POD30, POD90, and POD180 timepoints.

FIG. 4 shows MTT modulates keystone commensal genera following surgery. Mean relative abundance (±SD) of Collinsella and Akkermansia over time in control and MTT-treated patients. MTT is associated with increased abundance of both genera by POD14, with enrichment evident through POD30. *P<0.05 for between-group comparisons at indicated time points.

FIGS. 5A-5C show MTT modulates key commensal and pathobiont genera over time. Abundances of predominant genera (mean±standard deviation) in non-MTT controls and MTT recipients at baseline, inpatient, POD14, POD30, POD90, and POD180; (FIG. 5A) Relative abundances of Bacteroides and Phocaeicola, (FIG. 5B) Relative abundances of Blautia, Ruminococcaceae, and Lachnospiraceae; (FIG. 5C) Relative abundances of Enterococcus spp, and Streptococcus spp.

FIG. 6 shows MTT restores short-chain fatty acid (SCFA) production. Fecal concentrations (mean±SD) of butyrate, acetate, and propionate at baseline and POD14 in patients receiving MTT and non-MTT controls. SCFA levels decline after surgery in non-MTT controls and are significantly higher in MTT recipients by POD14, consistent with recovery of microbial fermentative function. *P<0.05; **P<0.005 for between-group comparisons at POD14.

FIGS. 7A-7C show gating strategies for identification of innate immune subsets in MTT samples. Density and contour plots illustrate the sequential gating strategy used to identify peripheral blood innate immune cell populations in MTT recipients. (FIG. 7A) Neutrophil and myeloid-derived suppressor cell (MDSC) panel. (FIG. 7B) Monocyte subsets. (FIG. 7C) NK, NKT, and dendritic cell (DC) populations. Arrows indicate successive stages of the gating hierarchy, depicting the structured progression from CD45⁺ viable singlets to defined immune subsets. Density and contour plots were generated from a minimum of 100,000 CD45⁺ live singlet events per sample and are representative of pooled donor data (n=6), ensuring robust representation of each immune population.

FIGS. 8A-8L show MTT induces temporally ordered innate and adaptive immune remodeling following colorectal surgery. (FIG. 8A) Fold change from baseline (POD1 MTT baseline samples) in neutrophil subsets at POD3 following MTT, including immature, activated, migratory, granule-releasing, and immune-associated phenotypes, quantified by spectral flow cytometry. These changes reflect increased neutrophil heterogeneity consistent with coordinated innate immune remodeling early after surgery. (FIG. 8B) Fold change from baseline (POD1 MTT baseline samples) in monocyte and innate immune subsets at POD3 following MTT, including myeloid-derived suppressor cells (MDSCs), natural killer (NK) cells, and dendritic cell populations. No significant changes were observed in conventional monocyte or dendritic cell subsets at this time point. (FIG. 8C) Fold change from baseline (POD1 MTT baseline samples) in adaptive immune subsets at POD30 following MTT, including CD4⁺ and CD8⁺ effector T cells, early and intermediate CD4⁺ effector populations, and regulatory T cells (Tregs), demonstrating later adaptive immune remodeling consistent with immune resolution. (FIG. 8D) Uniform Manifold Approximation and Projection (UMAP) visualization of neutrophil populations at POD1 and POD3 following MTT, colored by unsupervised cluster assignment. (FIG. 8E) Differences in neutrophil cluster proportions at POD3 relative to POD1 following MTT, expressed as changes in the proportion of each cluster relative to the total neutrophil population. (FIG. 8F) Heatmap showing normalized marker expression profiles defining neutrophil clusters, including markers associated with activation, degranulation, adhesion, migration, and maturation. (FIG. 8G) UMAP visualization of monocyte and dendritic cell populations at POD1 and POD3 following MTT, colored by unsupervised cluster assignment. (FIG. 8H) Differences in monocyte and dendritic cell cluster proportions at POD3 relative to POD1 following MTT, expressed as changes in the proportion of each cluster relative to the total myeloid population. (FIG. 8I) Heatmap showing normalized marker expression profiles defining monocyte and dendritic cell clusters, including features associated with antigen presentation and myeloid suppression. (FIG. 8J) UMAP visualization of adaptive immune populations at POD1 and POD30 following MTT, colored by unsupervised cluster assignment. (FIG. 8K) Differences in adaptive immune cluster proportions at POD30 relative to POD1 following MTT, expressed as changes in the proportion of each cluster relative to the total CD4⁺ or CD8⁺ T cell populations, as indicated. (FIG. 8L) Heatmap showing normalized marker expression profiles defining adaptive immune clusters, including markers associated with effector, regulatory, and memory phenotypes. Unsupervised clustering and dimensionality reduction analyses (FIG. 8D-L) corroborate and extend frequency-based findings (FIG. 8A-C) by revealing coordinated shifts in immune phenotypic states across early innate and later adaptive phases. Data are presented as mean±SD. Statistical significance was assessed using paired within-MTT recipients (POD1 vs POD3; POD1 vs POD30); ns indicates not significant; *P<0.05.

FIG. 9 shows gating strategy for identification of adaptive immune subsets in MTT samples. Density plots depict the sequential gating strategy used to define T and B lymphocyte populations in peripheral blood samples from MTT recipients. Arrows indicate each stage of the gating workflow, highlighting transitions between defined cell populations within the T and B cell panels. Plots were generated from a minimum of 100,000 CD45⁺ live singlet events per sample, concatenated across donors (n=6), to ensure consistent representation of adaptive immune subsets.

FIG. 10 shows MTT is associated with shifts in memory B cell and plasmablast populations. Quantification of B cell subsets following MTT is consistent with relative stability in naïve B cells alongside changes within antigen-experienced compartments. Switched and unswitched memory B cell populations exhibited increases relative to POD1 MTT baseline samples (not statistically significant). A statistically significant increase in plasmablast frequency was observed, consistent with differentiation of memory B cells into antibody-secreting cells. These findings align with adaptive immune remodeling observed at POD30 (FIG. 8) and support coordinated restructuring of humoral compartments during postoperative recovery.

FIG. 11 shows a conceptual model of coordinated recovery following MTT. Colorectal surgery induces perioperative ecological perturbation marked by microbial community collapse, depletion of SCFAs and BCFAs, and disruption of immune homeostasis. Postoperative administration of MTT introduces targeted microbial reconstitution during a window of ecological vacancy. Longitudinal profiling demonstrates temporally ordered coupling between durable microbial engraftment, restoration of microbial metabolic function, and immune reprogramming spanning early innate and later adaptive phases. Convergence of these processes defines an integrated recovery-linked latent axis (Z2), reflecting coordinated biological reconstitution across systems.

### DETAILED DESCRIPTION

Provided herein are methods and compositions for aiding individuals that have undergone an intestinal surgery. Examples of intestinal surgery include, but are not limited to, those that can be used to treat diverticulitis, colorectal cancer, and inflammatory bowel disease (IBD). An intestinal surgery can include intestinal resection and reconnection (anastomosis). An individual can be prepared for the surgery by bowel preparation (BP), involving a mechanical purge, optionally with oral antibiotics paired with intravenous (IV) antibiotics administered during the surgery (Nichols et al., Annals of Surgery. 1973; 178 (4): 453-462. doi: 10.1097/00000658-197310000-00008).

The methods described herein can help reduce, in a patient that has had an intestinal surgery, the incidence of post-operative complications including delayed return of bowel function (ROBF), infections (including anastomotic leaks (AL) and surgical site infections (SSI)), hospital length of stay, morbidity, and/or mortality.

The compositions for use in the methods described herein include administering microbes (microbiota) recovered from feces of human donors to the site of a surgical incision in a subject that has recently undergone the surgery, and before the surgical incision has healed. This delivery of fecal microbes to a surgical site is not in agreement with any post-surgical standard of care for reduction of infection. The administration of fecal microbes to the gastrointestinal tract has been shown to treat certain states of dysbiosis; however, the gastrointestinal tract of a patient following an intestinal surgery, especially a surgery that includes intestinal resection and reconnection, has undergone severe trauma and is in a very different physiological state. In fact, the rationale for the standard inclusion of antibiotics into the BP prior to surgery is to decrease the burden of gut microbes that could provoke an inflammatory response upon contact with the intestinal wound or escape through the incision to cause a local or systemic infection. Thus, it was not predictable that administering to a surgical site a composition that included fecal microbes would effectively treat a patient. Contrary to conventional wisdom, the inventors proceeded to use compositions of fecal microbes as described herein and found that administration of fecal microbes improved patient outcomes.

As used herein, the term “fecal microbes” refers to microorganisms that are present in the gut, intestine, colon, or feces of a normal healthy adult human. Such a composition may be prepared by processing fecal material. As used herein, the term “fecal material” refers to human stool. Unprocessed fecal material contains non-living material and biological material. The “non-living material” refers to the non-living material in fecal material, and may include, but is not limited to, dead bacteria, shed host cells, proteins, carbohydrates, fats, minerals, mucus, bile, undigested fiber and other foods, and other compounds resulting from food and metabolic ingestion and waste products and partial or complete digestion of food materials. “Non-living material” does not include an excipient, e.g., a pharmaceutically inactive substance, such as a cryoprotectant or lyoprotectant, added to a processed fecal material. “Biological material” refers to the living material, also referred to as microorganisms, in fecal material (e.g., all possible microorganisms present in a human gut), and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. As used herein, a “fecal extract” is a composition derived from a fecal sample, but includes predominantly biological material, including bacteria, and little non-living material, e.g., undigested fiber and other foods.

A composition described herein can be liquid or freeze-dried. As used herein, “liquid” refers to a composition having the characteristics described herein and further having an aqueous component, typically water, milk, or another liquid carrier. As used herein, “freeze-dried” refers to a composition having the characteristics described herein and further having a small amount of moisture dictated by the atmosphere of the preparation room, substantially no water present, and in one embodiment, no detectable water. Methods for freeze-drying a composition are known and routinely used. The word freeze-drying is used synonymously with lyophilization. A method for freeze-drying a composition may include one or more pretreatments (e.g., concentrating, addition of a cryoprotectant or lyoprotectant, increasing the surface area of a composition), freezing the composition, and drying (e.g., exposing the composition to a reduced atmospheric pressure to result in sublimation of the water present in the composition).

Prokaryotic cells that may be present in a composition described herein include the normal members of the intestinal microbiota present in any healthy human. Examples of prokaryotic cells that may be present include members of the class Actinobacteria, such as the subclass Actinobacteridae and subclass Coriobacteridae. Examples of the subclass Actinobacteridae include members of the order Actinomycetales, and members of the order Bifidobacteriales. Members of the order Bifidobacteriales include members of the family Bifidobacteriaceae. Examples of the subclass Coriobacteridae include members of the order Coriobacteriales. Members of the order Coriobacteriales include members of the family Coriobacteriaceae. Members of the family Coriobacteriaceae include the genus Collinsella.

Other examples of prokaryotic cells include members of the phylum Bacteroidetes, such as class Bacteroidia. Members of class Bacteroidia include order Bacteroidales. Members of order Bacteroidales include members of the family Bacteroidaceae (such as members of the genus Bacteroides), members of the family Porphyromonadaceae, members of the family Prevotellaceae (such as members of the genus Prevotella), and members of the family Rikenellaceae.

Other examples of prokaryotic cells include members of the phylum Firmicutes, such as class Bacilli, Clostridia, Erysipelotrichi, and Negativicutes.

Examples of the class Bacilli include members of the order Bacillales (including members of the family Paenibacillaceae and members of the family Planococcaceae) and the order Lactobacillus (including members of the family Aerococcaceae, Enterococcaceae, Lactobacillusceae, and Streptococcaceae). Examples of the family Enterococcaceae include members of the genus Enterococcus. Examples of the family Streptococcaceae include members of the genus Streptococcus.

Examples of the class Clostridia include members of the order Clostridiales, and examples of the order Colstridiales include the family Catabacteriaceae, Peptococcaceae, Peptostreptococcaceae, Ruminococcaceae (also known as the family Oscillospiraceae), Clostridiaceae, Eubacteriaceae, Lachnospiraceae, and Peptoniphilaceae. Examples of the family Peptostreptococcaceae include members of the genera Peptostreptococcus and Intestinibacter. Examples of the family Ruminococcaceae include members of the genera Ruminococcus, Faecalibacterium, and Butyricicoccus. Examples of the family Lachnospiraceae include members of the genera Blautia, Coprococcus, Dorea, Lachnospira, Oribacterium, and Roseburia. Examples of the family Peptoniphilaceae include members of the genus Parvimonas. Members of the family Eubacteriaceae include members of the genus Eubacterium.

Examples of the class Erysipelotrichi include members of the family Erysipelotrichaceae.

Examples of the class Negativicutes include members of the family Veillonellaceae. Other examples of the order Bacillales include Bacillales Family XI. Incertae Sedis, and Bacillaceae 1. Other examples of the order Clostridiales include Clostridiales Family XI. Incertae Sedis, Clostridiales Family XIII. Incertae Sedis, and Clostridiaceae. Other examples of prokaryotic cells include members of the phylum Proteobacteria, such as class Alphaproteobacteria, Betaproteobacteria, Deltaproteobacteria, Epsilonproteobacteria, and Gammaproteobacteria. Examples of the class Alphaproteobacteria include members of the order Rhizobiales, and examples of members of the order Rhizobiales includes members of the family Rhodobiaceae, members of the family Brucellaceae, and members of the family Hyphomicrobiaceae. Examples of the class Betaproteobacteria include members of the order Burkholderiales, and examples of members of the order Burkholderiales include members of the family Alcaligenaceae, members of the family Burkholderiaceae, and members of the family Sutterellaceae. Examples of the class Deltaproteobacteria include members of the order Desulfovibrionales, and examples of members of this order include members of the family Desulfovibrionaceae and Desulfomicrobiaceae. Examples of the class Epsilonproteobacteria include members of the order Desulfobacterales, and examples of members of this order include members of the family Desulfobacteraceae. Examples of the class Gammaproteobacteria includes members of the order Alteromonadales and Enterobacteriales. Examples of members of the order Alteromonadales include members of the family Shewanellaceae, and examples of members of the order Enterobacteriales include members of the family Enterobacteriaceae.

Other examples of prokaryotic cells include members of the phylum Mycoplasmatota, formerly Tenericutes. Examples of members of the phylum Mycoplasmatota include members of the class Mollicutes. Examples of the class Mollicutes include members of the order Entomoplasmatales, and members of the order Entomoplasmatales include members of the family Spiroplasmataceae.

Other examples of prokaryotic cells include members of the class Verrucomicrobiae. Examples of members of the class Verrucomicrobiae include members of the order Verrucomicrobiales, and examples of members of the order Verrucomicrobiales includes members of the family Verrucomicrobiaceae and the family Akkermansiaceae. Members of the family Akkermansiaceae include the genus Akkermansia. Other examples of prokaryotic cells include members of the family Fusobacteriaceae.

In one embodiment a composition described herein may include prokaryotic bacteria that are members of at least 1 phylum, at least 2 phyla, at least 3 phyla, at least 4 phyla, at least 5 phyla, at least 6 phyla, at least 7 phyla, at least 8 phyla, at least 9 phyla, or at least 10 phyla. In one embodiment a composition of the present disclosure may include prokaryotic bacteria that are members of at least 1 class, at least 2 classes, at least 3 classes, at least 4 classes, at least 5 classes, at least 6 classes, or at least 7 classes. In one embodiment a composition of the present disclosure may include prokaryotic bacteria that are members of at least 1 order, at least 2 orders, at least 3 orders, at least 4 orders, a least 5 orders, at least 6 orders, or at least 7 orders. In one embodiment a composition of the present disclosure may include prokaryotic bacteria that are members of at least 1 family, at least 2 families, at least 3 families, at least 4 families, at least 5 families, at least 6 families, at least 7 families. In one embodiment a composition of the present disclosure may include at least 5, at least 10, at least 20, or at least 30 different genera of prokaryotic bacteria. In one embodiment a composition of the present disclosure may include at least 10, at least 50, at least 100, at least 200, at least 300, at least 400, at least 500, at least 600, at least 700, at least 800, at least 900, or at least 1000 different species of prokaryotic bacteria.

In one embodiment a freeze-dried composition described herein includes, when reconstituted with water such as a buffer, no greater than 0.05%, no greater than 0.1%, no greater than 0.2%, no greater than 0.3%, no greater than 0.4%, no greater than 0.5%, no greater than 0.6%, no greater than 0.7%, no greater than 0.8%, no greater than 0.9%, no greater than 1%, no greater than 2%, no greater than 3%, no greater than 4%, no greater than 5%, no greater than 6%, no greater than 7%, no greater than 8%, no greater than 9% or no greater than 10% weight of non-living material/weight biological material (wt/wt). In one embodiment, the amount of non-living material in a composition of the present disclosure is undetectable using currently available techniques. For instance, living material can be stained for biological activity, electron transport, DNA, and RNA, and DNA and RNA for specific genes.

In one embodiment, the fecal material present in a liquid composition described herein, and the fecal material present in a freeze-dried composition described herein when reconstituted with water, does not include particles (e.g., particles of non-living material and/or particles of biological material) having a size of greater than 2.0 millimeters (mm), greater than 1.0 mm, greater than 0.7 mm, greater than 0.65 mm, greater than 0.6 mm, greater than 0.55 mm, greater than 0.5 mm, greater than 0.4 mm, greater than 0.3 mm, greater than 0.25 mm, greater than 0.212 mm, greater than 0.180 mm, greater than 0.150 mm, greater than 0.125 mm, greater than 0.106 mm, greater than 0.090 mm, greater than 0.075 mm, greater than 0.063 mm, greater than 0.053 mm, greater than 0.045 mm, greater than 0.038 mm, greater than 0.032 mm, greater than 0.025 mm, greater than 0.020 mm, or greater than 0.01 mm. Non-fecal material present in a composition may include particles having a size of greater than 2.0 mm, greater than 1.0 mm, greater than 0.7 mm, greater than 0.65 mm, greater than 0.6 mm, greater than 0.55 mm, greater than 0.5 mm, greater than 0.4 mm, greater than 0.3 mm, greater than 0.25 mm, greater than 0.212 mm, greater than 0.180 mm, greater than 0.150 mm, greater than 0.125 mm, greater than 0.106 mm, greater than 0.090 mm, greater than 0.075 mm, greater than 0.063 mm, greater than 0.053 mm, greater than 0.045 mm, greater than 0.038 mm, greater than 0.032 mm, greater than 0.025 mm, greater than 0.020 mm, or greater than 0.01 mm. In one embodiment, the fecal material present in a composition of the present disclosure consists of, or consists essentially of, particles of non-living material and/or biological material having a size that will pass through a sieve having a sieve size of 2.0 mm, 1.0 mm, 0.7 mm, 0.65 mm, 0.6 mm, 0.55 mm, 0.5 mm, 0.4 mm, 0.3 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm. Thus, in such an embodiment, the fecal material present in a composition has a size that is less than or equal to 2.0 mm, less than or equal to 1.0 mm, less than or equal to 0.7 mm, less than or equal to 0.65 mm, less than or equal to 0.6 mm, less than or equal to 0.55 mm, less than or equal to 0.5 mm, less than or equal to 0.4 mm, less than or equal to 0.3 mm, less than or equal to 0.25 mm, less than or equal to 0.212 mm, less than or equal to 0.180 mm, less than or equal to 0.150 mm, less than or equal to 0.125 mm, less than or equal to 0.106 mm, less than or equal to 0.090 mm, less than or equal to 0.075 mm, less than or equal to 0.063 mm, less than or equal to 0.053 mm, less than or equal to 0.045 mm, less than or equal to 0.038 mm, less than or equal to 0.032 mm, less than or equal to 0.025 mm, less than or equal to 0.020 mm, or less than or equal to 0.01 mm. The sieve size may be based on the US Standard sieve sizes of, for instance, 10, 18, 35, 60, 70, 80, 100, 120, 140, 170, 200, 230, 270, 325, or 400.

A composition of the present disclosure can include a flavor agent. As used herein, a “flavor agent” or “flavoring agent” is a compound or composition that can be added to a composition described herein, e.g., a liquid composition or a freeze-dried composition, to alter the flavor of the liquid composition, or to alter the flavor of a freeze-dried composition after it is reconstituted with an aqueous solution. A flavor agent can be liquid or powder. Examples of flavor agents include, but are not limited to, chocolate flavor, peppermint, methyl salicylate (wintergreen), or orange flavoring. Liquid and powder forms of chocolate flavor are commercially available.

A composition of the present disclosure may optionally include a cryoprotectant, a lyoprotectant, or both a cryoprotectant and a lyoprotectant. A cryoprotectant is a compound that maintains the viability of fecal microbes during freezing. A lyoprotectant is a compound that maintains the viability of fecal microbes during drying. Cryoprotectants and lyoprotectants are known in the art and used routinely to protect microbes when exposed to freezing and/or drying conditions. Examples include, but are not limited to, amino acids such as alanine, glycine, proline; simple sugars such as sucrose, glucose, lactose, ribose, mannitol, and trehalose; and other compounds such as dimethyl sulfoxide (DMSO), and glycerol. The amount of cryoprotectant and/or lyoprotectant present in a composition described herein may vary depending on the cryoprotectant and/or lyoprotectant used, the temperature to be used for freezing (e.g., −20° C., −80° C., or a different temperature), and the conditions used for drying. The amount of cryoprotectant and/or lyoprotectant that can be used is known to the skilled person or may be easily determined using routine experimentation. In one embodiment, a composition of the present disclosure may include trehalose or mannitol at a concentration of about 8% when prepared for the product to be administered orally, for instance via nasogastric or nasojejunal tube, or endoscopically.

A freeze-dried composition described herein includes a cryoprotectant and/or lyoprotectant. Cryoprotectants and lyoprotectants useful in freeze-drying microbes are known and include, for instance, amino acids such as alanine, glycine, proline; simple sugars such as sucrose, glucose, lactose, ribose, mannitol, and trehalose; other compounds such as dimethyl sulfoxide (DMSO), glycerol, casein hydrolysate, sucrose, gelatin, non-fat skim milk, starch hydrolysate, fetal calf serum, bovine serum albumin, or combinations of 1, 2, 3, 4, or more of the above cryoprotectants and lyoprotectants. Other cryoprotectants and lyoprotectants are also known. A cryoprotectant useful herein maintains the viability of fecal microbes when subjected to freeze-drying conditions, milling or grinding, and/or when stored as a freeze-dried composition. A lyoprotectant is a compound that maintains the viability of fecal microbes during drying. Milling, also referred to as grinding, is a process that physically changes a material into smaller particles. Methods for milling freeze-dried compositions are known to the skilled person, and can occur at various temperatures, e.g., at or below 0° C., or above 0° C. A cryoprotectant and/or lyoprotectant useful herein results in a freeze-dried composition that is friable. As used herein, a “friable” composition refers to a composition that can be easily milled to result in a fine powder. In one embodiment, a freeze-dried composition described herein that is friable is one that results in a powder that can be subsequently used to produce a tablet. In one embodiment, a useful powder may have size, density, flow, and compression characteristics suitable for production of tablets or encapsulation, or double encapsulation.

Useful cryoprotectants and/or lyoprotectants include those that result in a composition that is friable, does not crystallize during freeze-drying, and maximize survival of microbes. The amount of cryoprotectant and/or lyoprotectant that can be used is known to the skilled person or may be easily determined using routine experimentation. In one embodiment, a composition of the present disclosure may include trehalose or mannitol at a concentration of about 8% when prepared for use as a colonoscopic product. In one embodiment, more than one cryoprotectant may be used, such as the combination of sucrose (e.g., 5%) and skim milk (e.g., 5%), trehalose and skim milk (e.g., 5%), trehalose and dextran (e.g., 3-10%), trehalose and mannitol, or the combination of sucrose (e.g., 10%) and gelatin (e.g., 0.1%).

In one embodiment, a useful cryoprotectant and/or lyoprotectant is not sucrose alone, which may crystallize and harden during freeze-drying, or glycerol, which unexpectedly results in an oily and viscous composition upon freeze-drying with a fecal material as described herein.

The total cryoprotectant and/or lyoprotectant used to produce a freeze-dried composition may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% (vol/vol), or more of the final concentration of a mixture of fecal microbes and the cryoprotectant and/or lyoprotectant before freeze-drying the composition. For instance, to produce a composition having a cryoprotectant and/or lyoprotectant at a final concentration of 10%, equal volumes of a 20% solution of the cryoprotectant and/or lyoprotectant and a mixture of microbes derived from fecal material can be combined and mixed, and then freeze-dried.

In one embodiment a composition of the present disclosure, e.g., a freeze-dried composition, does not include pathogenic biological material. In one embodiment, fecal material is from a person, referred to herein as a donor, that has undergone a clinical evaluation, including medical history, a physical examination, and laboratory testing. The clinical evaluation may include, but is not limited to, risk of infectious agents, carriage of multi-drug-resistant organisms, presence of gastrointestinal co-morbidities, factors that can or do affect the composition of the intestinal microbiota, and systemic medical conditions.

Exclusion criteria regarding risk of infectious agents may include, but are not limited to, known viral infection with Hepatitis B, C or HIV; known exposure to HIV or viral hepatitis at any time; high risk behaviors including sex for drugs or money, any past use of intravenous drugs or intranasal cocaine, history of incarceration; tattoo or body piercing within 12 months; travel to areas of the world where risk of traveler's diarrhea is higher than the US; and current communicable disease, e.g., upper respiratory viral infection.

Exclusion criteria regarding gastrointestinal comorbidities include, but are not limited to, history of irritable bowel syndrome, wherein specific symptoms may include frequent abdominal cramps, excessive gas, bloating, abdominal distension, fecal urgency, diarrhea, constipation; history of inflammatory bowel disease such as Crohn's disease, ulcerative colitis, microscopic colitis, chronic diarrhea; chronic constipation or use of laxatives; history of gastrointestinal malignancy or known colon polyposis; history of any abdominal surgery, e.g., gastric bypass, intestinal resection, appendectomy, cholecystectomy, and the like; use of probiotics or any other over the counter aids used by the potential donor for purpose of regulating digestion, but yogurt and kefir products may be allowed if taken merely as food rather than nutritional supplements.

Exclusion criteria regarding factors that can, or do affect, the composition of the intestinal microbiota include, but are not limited to, antibiotics for any indication within the preceding 6 months, and any prescribed immunosuppressive or anti-neoplastic medications.

Exclusion criteria regarding systemic medical conditions include, but are not limited to, established or emerging metabolic syndrome, where criteria used for definition here are stricter than established criteria, including history of increased blood pressure, history of diabetes or glucose intolerance; known systemic autoimmunity, e.g., connective tissue disease, multiple sclerosis; known atopic diseases including asthma or eczema, chronic pain syndromes including fibromyalgia, chronic fatigue syndrome; ongoing (even if intermittent) use of any prescribed medications, including inhalers or topical creams and ointments; neurologic, neurodevelopmental, and neurodegenerative disorders including autism, Parkinson's disease.

Exclusion criteria on physical examination may include, but are not limited to, general, such as body mass index <30 kg/m², central obesity defined by waist: hip ratio >0.90 (male) and >0.85 (female); blood pressure >135 mmHg systolic and >85 mmHg diastolic; skin—presence of a rash, tattoos or body piercing placed within a year, jaundice; enlarged lymph nodes; wheezing on auscultation; hepatomegaly or stigmata of liver disease; swollen or tender joints; muscle weakness; abnormal neurologic examination.

Exclusion criteria on laboratory testing may include, but is not limited to, detection of Clostridium difficile toxin B, Escherichia coli O157: H7, Shiga toxins, Enteropathogenic E. coli (EPEC), Salmonella, Shigella, Yersinia, Campylobacter, Pleisomonas, and Vibrio; Giardia, Cryptosporidium, Cyclospora, and Cystoisospora (previously Isospora); Rotavirus, Norovirus I and II, and adenovirus by PCR; detection of multi-drug resistant organisms, including, but not limited to, MRSA, ESBL, CRE, VRE using PCR or culture in selective media; positive screening for any circulating viral pathogens, including HIV 1 and 2, Hepatitis A, Hepatitis B, Hepatitis C, Cytomegalovirus, Covid, Epstein-Barr Virus; any abnormal liver function tests including alkaline phosphatase, aspartate aminotransaminase, alanine aminotransferase; raised serum triglycerides >150 mg/dL; HDL cholesterol <40 mg/dL (males) and <50 mg/dL (females); high sensitivity CRP >2.4 mg/L; raised fasting plasma glucose (>100 mg/dL).

In one embodiment, a donor has a higher microbiota content of short-chain fatty acid (SCFA)-producing bacteria. Examples of SCFA-producing bacteria include, but are not limited to, members of the family Lachnospiraceae, including members of the genera Blautia, Coprococcus, Dorea, Lachnospira, Oribacterium, and Roseburia, and members of the family Ruminococcaceae (also known as the family Oscillospiraceae), such as members of the genera Ruminococcus and Faecalibacterium.

In one embodiment, a donor has a higher microbiota content of bacteria producing oligosaccharides and organic acids that serve as substrates for downstream production of SCFAs such as lactate, formate, and other intermediates that feed pathways supporting butyrate and propionate production by fermentative taxa. Examples of bacteria that provide feedstock for SCFA synthesis include, but are not limited to, members of the family Akkermansiaceae, and members of the family Coriobacteriaceae. Members of the family Akkermansiaceae, include, but are not limited to the genus Akkermansia. Members of the genus Akkermansia include, but are not limited to, Akkermansia mucinophila. Members of the family Coriobacteriaceae include, but are not limited to, members of the genus Collinsella.

The compositions of the present disclosure may be included in a diversity of pharmaceutically acceptable formulations. In one embodiment, a formulation may be a liquid composition. Liquid compositions include, but are not limited to, solutions, suspensions, dispersions, and the like. In one embodiment, a formulation may be a solid composition. Solid compositions include, but are not limited to, powder (e.g., a friable freeze-dried powder), granule, compressed tablet, pill, capsule, chewing gum, microsphere, wafer, and the like. Those formulations may include a pharmaceutically acceptable carrier to render the composition appropriate for administration to a subject. As used herein “pharmaceutically acceptable carrier” includes pharmacologically inactive compounds compatible with pharmaceutical administration. A pharmaceutically acceptable carrier can be, and typically is, United States Pharmacopeia (USP) grade. Examples of pharmaceutically acceptable carriers include flavor agents and gut-transit protectants. The compositions of the present disclosure may be formulated to be compatible with its intended route of administration. In one embodiment, a composition is formulated for oral administration as a liquid.

A composition of the present disclosure may be administered by any method suitable for depositing in the gastrointestinal tract of a subject. Examples of routes of administration include oral administration (e.g., by a solid such as a pill, tablet, or capsule), or by liquid, via intubation through the nose or the mouth (e.g., by nasogastric tube or nasal jejunal tube, upper endoscopy, upper push enteroscopy, balloon enteroscopy), or rectal administration (e.g., by suppository, enema, flexible sigmoidoscopy, or colonoscopy). In embodiments where a liquid form of the composition is delivered to a subject, the freeze-dried composition can be reconstituted with an aqueous solution, such as by adding water, saline, milk, or plant-based milk, a flavor agent, and/or a gut-transit protectant, or by exposing the freeze-dried composition to a body fluid. A gut-transit protectant may include an acid suppressant medication, such as antacid, an H₂-blocker, a proton pump inhibitor, or potassium-competitive acid blocker. In one embodiment, a composition is formulated for oral administration as a liquid that is swallowed by the subject. Such a composition is also referred to herein as an oral liquid preparation.

For therapeutic use in the method of the present disclosure, a composition described herein may be conveniently administered in a form containing one or more pharmaceutically acceptable carriers. Suitable carriers are well known in the art and vary with the desired form and mode of administration of the composition. For example, they may include diluents or excipients such as fillers, binders, wetting agents, disintegrators, surface-active agents, glidants, lubricants, and the like. Typically, the carrier may be a solid (including powder), liquid, or combinations thereof. Each carrier is preferably “acceptable” in the sense of being compatible with the other ingredients in the composition and not injurious to the subject. The carrier is preferably biologically acceptable and inert, i.e., it permits the composition to maintain viability of the biological material until delivered to the appropriate site.

Compositions formulated for oral administration may include an inert diluent or an edible carrier. For purposes of oral therapeutic administration, in one embodiment a liquid composition, e.g., an oral liquid preparation, can include a flavor agent, a gut-transit protectant, or both a flavor agent and a gut-transit protectant. A flavor agent and/or a gut-transit protectant can be present in a freeze-dried composition that is reconstituted to result in a liquid composition, or a flavor agent and/or a gut-transit protectant can be added to the composition at the time of reconstitution. Flavor agents are described herein. A gut-transit protectant is a compound or composition that protects fecal microbes during gut transit, e.g., transit through the high acidity and digestive enzymes present in the stomach and/or intestine. Examples of gut-transit protectants include, but are not limited to, a dairy product such yogurt, a milk or powdered milk. Examples of milk include regular or chocolate milk, and whole milk or milk having reduced fat, such as 1% or 2% milk fat. Other examples of milk include a plant-based milk such as, but not limited to. almond, oat, and soy. Examples of powdered milk, also referred to as milk powder, dried milk, and dry milk, include but are not limited to nonfat skimmed milk, whole milk, buttermilk, and whey. Gut-transit protectants can be added to a liquid composition to a final concentration of at least 1% w/w, at least 5% w/w, at least 10% w/w, or at least 15% w/w, and no greater than 20% w/w, no greater than 15% w/w, no greater than 10% w/w, or no greater than 5% w/w. Exemplary ranges include but are not limited to at least 1% w/w to no greater than 20% w/w, or at least 5% w/w to no greater than 15% w/w. A gut-transit protectant may also include an acid suppressant medication, such as antacid, an H2-blocker, a proton pump inhibitor, or potassium-competitive acid blocker. In one embodiment, for the purpose of oral therapeutic administration a freeze-dried composition can be incorporated with excipients and used in the form of tablets, or capsules, e.g., gelatin or hypromellose capsules. Oral compositions can also be prepared by combining a composition of the present disclosure with a food. In one embodiment a food used for administration is chilled, for instance, ice cream or milk. Pharmaceutically compatible binding agents, and/or adjuvant materials can be included as part of the composition. The tablets, pills, capsules, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; or a sweetening agent such as sucrose or saccharin. Other ingredients may be added to a formulation to provide desired characteristics such as flow, compression, hardness, and taste.

In one embodiment, the freeze-dried composition may be present in a formulation that permits passage to the small intestine or colon. For instance, when the composition is to be administered orally using capsules, the dosage form may be formulated so the composition is not exposed to conditions prevalent in the gastrointestinal tract before the small intestine or colon, e.g., high acidity and digestive enzymes present in the stomach and/or intestine. In one embodiment, the dosage form may be formulated so the composition passes through the stomach and is released in conditions that include a pH of greater than 5.5, greater than 6, greater than 6.5, or greater than 7. In one embodiment an enteric coating is acid-resistant to protect the composition from the low pH of the stomach and break down when exposed to a pH greater than present in the stomach. The encapsulation of compositions in an enteric coating for therapeutic use is routine in the art. Materials used for enteric coatings include fatty acids, waxes, shellac, plastics, and plant fibers. Examples include, but are not limited to, methyl acrylate-methacrylic acid copolymers, cellulose acetate succinate, hydroxy propyl methyl cellulose phthalate, enteric coatings (hydroxypropyl methylcellulose (HPMC), hydroxy propyl methyl cellulose acetate succinatelate (PVAP), methyl methacrylate-methacrylic acid copolymers, cellulose acetate trimellitate, and sodium alginate. Encapsulation may include hard-shelled capsules, which may be used for dry, powdered ingredients, or soft-shelled capsules. Capsules may be made from aqueous solutions of gelling agents such as animal protein (e.g., gelatin), plant polysaccharides or derivatives like carrageenans and modified forms of starch and cellulose. Other ingredients may be added to a gelling agent solution such as plasticizers (e.g., glycerin and or sorbitol), coloring agents, preservatives, disintegrants, lubricants, and surface treatment. Enteric coated capsules can be co-combined to provide for release of the freeze-dried composition within the large bowel or colon.

Useful diluents include aqueous solutions that are routinely used for manipulating microbes, eukaryotic cells, and/or viruses. Useful diluents may include constituents to maintain physiological buffer, osmolarity, and the like. The diluent is preferably sterile and/or non-allergenic. An example of a diluent includes, but is not limited to, saline or phosphate buffered saline at pH 7. In one embodiment, 1 part donor feces may be combined with 5 parts diluent (e.g., 50 grams of donor feces may be combined with 250 ml diluent) and blended. In one embodiment, the oxygen in the blending chamber may be decreased or removed by purging with an inert gas such as nitrogen or argon prior to blending. Such anaerobic conditions may be useful to maintain viability of most anaerobic bacteria present in a colon. The sample may be blended multiple times and/or more diluent may be added until a consistency is achieved that will permit the following steps to occur. In one embodiment, anaerobic conditions are not used in steps following the blending. It was found that anaerobic conditions were not necessary in the steps following the blending, and this was unexpected and surprising since a substantial percentage of prokaryotic cells in fecal material are strict anaerobes, and exposure to oxygen kills them. After the blending, the solutions used for washing and resuspension did not need to be purged of oxygen, and manipulation of the microbiota in an oxygen free cabinet or glove box was not needed.

In one embodiment, a blended sample may be prepared by obtaining a fecal sample from an appropriate donor and blending with a diluent as described in Sadowsky et al. (WO 2012/122478, WO 2014/152484). In another embodiment, a composition may be prepared by obtaining a fecal sample from an appropriate donor and using ballistic disruption with a horizontal or vertical shaker, a suitable diluent and stainless steel beads of 3.2 mm in diameter. The mixture is shaken to break up the sample and the beads are removed from the suspension by filtration a stainless steel strainer. The suspension is centrifuged at a suitable speed to pellet the microbes, for instance, 500-1,000 rpm, the supernatant poured off and the resulting microbial fraction obtained by selective filtration.

Not all microbes and eukaryotic cells present in an individual's colon can be cultured, thus, in one embodiment conditions for preparing a composition of the present disclosure include the use of temperatures that decrease the replication of the microbes and eukaryotic cells. In one embodiment, the conditions used for preparation are maintained below 37° C. For instance, the conditions used for preparation are maintained at a temperature of no greater than 30° C., no greater than 20° C., no greater than 10° C., or no greater than 5° C. In one embodiment, conditions are used such that replication of the microbes and eukaryotic cells is undetectable, and preferably does not occur. When the conditions used to prepare a composition of the present disclosure include lower temperatures to minimize replication and cell death, the biological material present in a composition includes a population of microbes, eukaryotic cells, and viruses that is essentially identical to a population of microbes, eukaryotic cells, and viruses present in the colon or feces of a normal healthy human, e.g., the donor from whom the fecal sample was obtained. In one embodiment, the conditions used for preparation decrease exposure of the microbes and eukaryotic cells to oxygen, both before and after purification of microbiota.

As described in Sadowsky et al. (WO 2012/122478, WO 2014/152484) removal of non-living material may be achieved by selective filtration, e.g., passing the blended sample through a series of sieves with a sieve size of no greater than 2.0 mm, no greater than 1.0 mm, no greater than 0.5 mm, no greater than 0.25 mm, no greater than 0.212 mm, no greater than 0.180 mm, no greater than 0.150 mm, no greater than 0.125 mm, no greater than 0.106 mm, no greater than 0.090 mm, no greater than 0.075 mm, no greater than 0. 063 mm, no greater than 0.053 mm, no greater than 0.045 mm, no greater than 0.038 mm, no greater than 0.032 mm, no greater than 0.025 mm, no greater than 0.020 mm, no greater than 0.01 mm, or no greater than 0.2 mm. In one embodiment, the blended sample is prepared by passing it through a sieve with a sieve size of 0.25 mm and collecting the filtrate. In one embodiment, the blended sample is passed through sieves with progressively smaller sieve sizes until final passage through a sieve size of 0.25 mm. For instance, if a total of four sieves are used the sieve size of the first sieve may be 2 mm, followed by 1 mm, followed by 0.5 mm, and then followed by 0.25 mm. The final filtrate may be collected in a centrifuge tube, and centrifuged at a speed sufficient to pellet the biological material, for instance, 5,000×g for 10 minutes at 4° C. The supernatant is removed, the cells are resuspended in diluent, optionally centrifuged again, for instance at 5,000×g for 10 minutes at 4° C. The final supernatant is discarded, and the cells are resuspended in an aqueous solution (e.g., diluent). In one embodiment, the volume of the blended mixture is decreased through the steps of sieving and washing. For instance, in one embodiment, the volume is decreased to 14% of the volume used in the blending (e.g., from 250 mls to 35 mls). In one embodiment, the volume of the blended mixture is decreased through the steps of sieving and washing to result in at least 1×10¹⁰ cells in a volume that is subsequently administered to a subject. The final filtrate may also be collected in a centrifuge tube, washed, and the cells resuspended in an aqueous solution (e.g., diluent, cryoprotectant, and the like, or a combination thereof). In one embodiment, the volume of the blended mixture is decreased through the steps of sieving and washing to result in at least 1×10¹⁰ cells in a volume that is subsequently freeze-dried. Since most biological material is difficult or impossible to culture, a hemocytometer may be used to determine the number of cells. This process results in an extract of feces that is highly enriched for all colon microbiota that are able to pass through a sieve as described above, and can be centrifuged at 10,000×g for 10 minutes. As used herein, “enriched” refers to increasing the abundance of biological material relative to non-living material, such that biological material constitutes a significantly higher proportion compared to the fecal material before the enrichment. The term “enriched” refers to those situations in which a person has intervened to elevate the proportion of biological material.

The amount of aqueous solution added may be in an amount to result in a single dosage having an appropriate number of cells. In one embodiment, a single dosage may include at least 5×10¹⁰ cells, at least 5.5×10¹⁰ cells, at least 6×10¹⁰ cells, at least 6.5×10¹⁰ cells, at least 7×10¹⁰ cells, at least 7.5×10¹⁰ cells, at least 8×10¹⁰ cells, at least 8.5×10¹⁰ cells, at least 9×10¹⁰ cells, at least 9.5×10¹⁰ cells, at least 1×10¹¹ cells, at least 2.5×10¹¹ cells, at least 3×10¹¹ cells, at least 3.5×10¹¹ cells, at least 4×10¹¹ cells, at least 4.5×10¹¹ cells, at least 5×10¹¹ cells, or at least 5.5×10¹¹ cells, at least 6×10¹¹ cells, at least 6.5×10¹¹ cells, at least 7×10¹¹ cells, at least 7.5×10¹¹ cells, at least 8×10¹¹ cells, at least 8.5×10¹¹ cells, at least 9×10¹¹ cells, at least 9.5×10¹¹ cells, at least 1×10¹² cells, at least 1.5×10¹² cells, at least 2×10¹² cells, at least 2.5×10¹² cells, at least 3×10¹² cells, at least 3.5×10¹² cells, at least 4×10¹² cells, at least 4.5×10¹² cells, at least 5×10¹² cells, or at least 5.5×10¹² cells, at least 6×10¹² cells, at least 6.5×10¹² cells, at least 7×10¹² cells, at least 7.5×10¹² cells, or at least 8×10¹² cells. In one embodiment, a single dosage may include no greater than 9×10¹² cells. Since most biological material is difficult or impossible to culture, a hemocytometer may be used to determine the number of cells.

In one embodiment the resulting pellet may be suspended in half the original volume of diluent containing 10% glycerol. The sample may be used immediately, or may be frozen, for instance, at −80° C., for later use. When freezing, the sample may be left in a centrifuge tube, or may be in a different container. In one embodiment, the container is one that increases the surface area of the sample. For instance, the sample may be placed in a cryo bag. When the frozen sample is to be used, it may be thawed on ice and then transplanted into the recipient. It was found that freezing the compositions described herein did not result in destruction of its curative potential. In one embodiment the sample resulting from centrifugation may be processed for long term storage of 1 year or longer. The ability to store such a sample provides a level of flexibility that was not possible with other methods. For instance, it was necessary to quickly identify a donor, rapidly process a fecal sample from the donor, and use it immediately. Examples of useful processing methods include, but are not limited to, freezing, and freeze drying or lyophilization. Processing of a composition of the present disclosure may include the production of a powder following any drying procedure.

In some embodiments, making a composition disclosed herein can include at least four separation/washing steps. Typically, the final filtrate obtained after selective filtration, e.g., passing the blended sample through a series of sieves, is subjected to a force to cause separation of material from liquid and, after removal of the liquid, the resulting material is resuspended in diluent. This process of separation, removal of supernatant, and resuspension is repeated at least 3 additional times for a total of four separations and washes. The force to cause separation of material from liquid can be centrifugal force from centrifugation. The speed of a centrifugation step can be at any speed that causes the separation of material from liquid but does not cause loss of therapeutic efficacy of the composition in FMT or MTT. Care should be taken as damage of the fecal microbes can be increased at greater speeds, and the fecal microbes can be more difficult to resuspend. Examples of suitable conditions include speeds resulting in at least 3,000×g, at least 4,000 xg, or at least 5,000×g. Other examples of suitable conditions include speeds resulting in no greater than 9,000×g, no greater than 8,000×g, or no greater than 7,000×g. Suitable ranges include at least 3,000×g to no greater than 9,000×g, at least 4,000×g to no greater than 8,000×g, and at least 5,000×g to no greater than 7,000×g. Typically, the volume of diluent added back to the material after removal of the supernatant is reduced to cause concentration of the material. For instance, the diluent added back to the material can be one half the volume present before the separation step. In one embodiment, the material is concentrated to result in at least 5×10¹⁰ cells, at least 5.5×10¹⁰ cells, at least 6×10¹⁰ cells, at least 6.5×10¹⁰ cells, at least 7×10¹⁰ cells, at least 7.5×10¹⁰ cells, at least 8×10¹⁰ cells, at least 8.5×10¹⁰ cells, at least 9×10¹⁰ cells, at least 9.5×10¹⁰ cells, at least 1×10¹¹ cells, at least 2.5×10¹¹ cells, at least 3×10¹¹ cells, at least 3.5×10¹¹ cells, at least 4×10¹¹ cells, at least 4.5×10¹¹ cells, at least 5×10¹¹ cells, or at least 5.5×10¹¹ cells, at least 6×10¹¹ cells, at least 6.5×10¹¹ cells, at least 7×10¹¹ cells, at least 7.5×10¹¹ cells, at least 8×10¹¹ cells, at least 8.5×10¹¹ cells, at least 9×10¹¹ cells, at least 9.5×10¹¹ cells, at least 1×10¹² cells, at least 1.5×10¹² cells, at least 2×10¹² cells, at least 2.5×10¹² cells, at least 3×10¹² cells, at least 3.5×10¹² cells, at least 4×10¹² cells, at least 4.5×10¹² cells, at least 5×10¹² cells, or at least 5.5×10¹² cells, at least 6×10¹² cells, at least 6.5×10¹² cells, at least 7×10¹² cells, at least 7.5×10¹² cells, or at least 8×10¹² cells in a volume that is subsequently freeze-dried. In one embodiment, the material is concentrated to result in no greater than 9×10¹² cells in a volume that is subsequently freeze-dried.

In some embodiments, making a composition disclosed herein can include homogenizing a sample. As used herein, “homogenizing” refers to conditions that promote the break-up of non-living and living material present in a fecal sample and increase the release of living biological material from the non-living material. A fecal sample can be present in a container in which homogenization can occur. An example of such a container is a flexible sterile plastic bag. In one embodiment, the oxygen in a container may be decreased or removed by purging with an inert gas such as nitrogen or argon prior to homogenization. Such anaerobic conditions may be useful to maintain viability of strict anaerobic bacteria present in a colon. The sample may be homogenized multiple times and/or additional diluent may be added until a consistency is achieved that will permit the following steps to occur. After the homogenizing, the solutions used for washing and resuspension can be treated to remove oxygen. Manipulation of the microbiota may occur in an oxygen free cabinet or anaerobic glove box chamber.

In one embodiment, a method disclosed herein (optionally referred to as a pressurized filter press method) can use a container such as a flexible sterile plastic bag that is impervious to liquid. Examples of such containers are described in, but not limited to, U.S. Pat. No. 5,180,229, US 2024/0009908, and US 2024/0017888, and those available from WHIRL-PAK® Filtration Group (Pleasant Prairie, WI). Care should be taken during the methods described herein to reduce exposure to oxygen, and the transfer can include using nitrogen gas to replace any air that may be present.

A cell extraction buffer is added to the fecal sample. The use of gelatin in combination with ammonium phosphate aids in the release of biological material from the non-living material present in a fecal sample, accordingly, in some embodiments, a cell extraction buffer includes gelatin and ammonium phosphate.

In one embodiment a cell extraction buffer includes, but is not limited to a buffer, or both a buffer and a gelatin. In one embodiment, 1 part donor feces may be combined with 5 parts cell extraction buffer (e.g., 50 grams of donor feces may be combined with 250 ml cell extraction buffer) and homogenized. An example of a buffer includes, but is not limited to, (NH₄)₂HPO₄. The buffer, for instance (NH₄)₂HPO₄, can be present at a concentration of at least 0.01 M, at least 0.05 M, or at least 0.1 M, and at a concentration of no greater than 1 M, no greater than 0.5 M, or no greater than 0.1 M. Examples of ranges of a buffer include, but are not limited to, 0.01 M to 1 M, and 0.05 M to 0.5 M. Gelatin is typically a mixture of peptides and proteins commonly derived from collagen. In one embodiment, the gelatin can be partially hydrolyzed. In one embodiment, the gelatin can be USP grade. Gelatin can be present at a concentration of at least 0.01% volume/volume (v/v), at least 0.05% v/v, or at least 0.1% v/v, and at a concentration of no greater than 1% v/v, no greater than 0.5% v/v, or no greater than 0.1% v/v. In one embodiment, a 10% v/v gelatin solution in water is adjusted to pH 10.0 and is partially hydrolyzed by autoclaving for 10 min at 15 PSI (Kingsley and Bohlool, Appl Environ Microbiol. 1981 August; 42 (2): 241-8. doi: 10.1128/aem.42.2.241-248.1981). Examples of ranges of gelatin in a cell extraction buffer include, but are not limited to, 0.01% v/v to 1% v/v, and 0.05% v/v to 0.5% v/v.

A cell extraction buffer can also include other components, including but not limited to a nonionic surfactant and emulsifier. Examples of a nonionic surfactant and emulsifier include, but are not limited to, polysorbate 80 (available under the trade name TWEEN-80®). A polysorbate 80 can be present at a concentration of at least 0.001%, at least 0.005%, or at least 0.01%, and at a concentration of no greater than 0.1%, no greater than 0.05%, or no greater than 0.01%.

The fecal sample in the container is subjected to conditions that homogenize the sample. In one embodiment, such conditions can be obtained through use of a paddle homogenizer, also known in the art as a stomacher. Typically, the sample is homogenized until it is a slurry, e.g., a mixture of solids suspended in a liquid. The slurry is then filtered with a pressurized filter press. An example of a pressurized filter press useful in the method includes a cylinder, a lid, and a plunger. The side of the plunger in contact with the surface of the cylinder fits tightly to prevent seepage of the slurry, and the surface of the plunger in contact with the slurry includes at least one filter medium. An example of a pressurized filter press is a French Press, modified to include a filter as described herein.

The filter medium present at the surface of the plunger in contact with the slurry includes at least one, at least two, at least three, or at least four filters. In one embodiment, the surface of the plunger in contact with the slurry includes no greater than six filters. The one or more filters can be described by sieve size, also referred to as pore size, in millimeters (mm) or sieve size based on the US Standard sieve sizes. The sieve size of one or more of the filters can be no greater than 2 mm, no greater than 1 mm, no greater than 0.7 mm, no greater than 0.65 mm, no greater than 0.6 mm, no greater than 0.55 mm, no greater than 0.5 mm, no greater than 0.25 mm, no greater than 0.212 mm, no greater than 0.18 mm, no greater than 0.15 mm, no greater than 0.125 mm, no greater than 0.106 mm, no greater than 0.09 mm, no greater than 0.075 mm, no greater than 0.063 mm, no greater than 0. 053 mm, no greater than 0.045 mm, no greater than 0.038 mm, no greater than 0.033 mm, no greater than 0.025 mm, no greater than 0.02 mm, or no greater than 0.01 mm. The sieve size of one or more filters can be no less than 0.212 mm, no less than 0.25 mm, no less than 0.3 mm, no less than 0.400 mm, no less than 0.5 mm, no less than 0.55 mm, no less than 0.6 mm, no less than 0.65 mm, no less than 0.7 mm, or no less than 0. mm. Sieve size of one or more of the filters can be, when described as US Standard sieve sizes, no greater than 400, no greater than 325, no greater than 270, no greater than 230, no greater than 200, no greater than 170, no greater than 140, no greater than 120, no greater than 100, no greater than 80, no greater than 70, no greater than 60, no greater than 35, no greater than 18, or no greater than 10. In one embodiment, the filter medium present at the surface of the plunger in contact with the slurry includes at least one, at least two, at least three, or at least four filters, each of which has a sieve size of no greater than 0.15 mm or no greater than US Standard sieve size 100. In one embodiment, the filter medium present at surface of the plunger in contact with the slurry includes at least four filters, each of which has a sieve size of 0.15 mm or US Standard sieve size 100.

Pressure can be applied manually to push the liquid component and biological material through the filter while preventing passage of the non-living material and biological material that exceeds the pore size of the smallest filter. The liquid component containing biological material is removed (filtrate) and the solids (unfiltered material) resuspended in a diluent, such as a saline solution. Saline solutions useful in maintaining the viability of biological material present in a fecal sample are known, and in one embodiment includes phosphate buffered saline at pH 7.0. This suspension is then filtered with the pressurized filter press to yield a second liquid component (second filtrate) containing biological material. The first and second liquid components are combined in a container suitable for centrifugation. The container can be purged with an inert gas or gas mixture, such as nitrogen gas, to maintain conditions for anaerobes. The force of a centrifugation step can be at any speed that causes the separation of biological material from liquid but does not reduce ease of resuspension in a suitable buffer, and does not cause loss of therapeutic efficacy of the composition in MTT. Care should be taken as damage of the fecal microbes can be increased at greater speeds, and the fecal microbes can be more difficult to resuspend. Examples of suitable conditions include speeds resulting in at least 3,000×g, at least 4,000×g, or at least 5,000×g. Other examples of suitable conditions include speeds resulting in no greater than 9,000×g, no greater than 8,000×g, or no greater than 7,000×g. Suitable ranges include at least 3,000×g to no greater than 9,000×g, at least 4,000×g to no greater than 8,000×g, and at least 5,000×g to no greater than 7,000×g. Typically, the volume of diluent added back to the biological material after removal of the supernatant is reduced to cause concentration of the material. For instance, the diluent added back to the material can be one half the volume present before the separation step. The washing procedure of centrifugation followed by removal of the supernatant and resuspending the pellet in a reduced volume of diluent is repeated at least once for a total of four washes.

In another embodiment, a method disclosed herein (optionally referred to as a linear filtration method) can use a container such as a flexible sterile plastic bag that is impervious to liquid, and has been further modified to include one or more linear filters. In one embodiment, a linear filter in a container is sealed to both sides and bottom of the container, resulting in two compartments that share one wall that is the filter. For instance, a container such as those described in, but not limited to, U.S. Pat. No. 5,180,229, US 2024/0009908, and US 2024/0017888, can be modified to include one or more filters. Useful containers containing a filter are available from WHIRL-PAK® Filtration Group (e.g., WHIRL-PAK® Filter Sterilized Bags). A fecal sample can be placed on either side, and the liquid component with biological material will transfer through the filter, while the non-living materials remain separate and contained.

In another embodiment, a fecal sample can be placed in a bag that includes one or more walls that are made of a linear filter. The bag can be sealed and then placed in a container such as a flexible sterile plastic bag that is impervious to liquid. For instance, a container such as those described in, but not limited to, U.S. Pat. No. 5,180,229, US 2024/0009908, and US 2024/0017888. Useful containers containing a filter are available from WHIRL-PAK® Filtration Group (e.g., WHIRL-PAK® Filter Sterilized Bags). The liquid component of the fecal sample with biological material will transfer through the filter, while the non-living materials remain separate and contained. The inventors have found that the method of using this double filtration system resulted in surprisingly efficient production of a homogenized sample that can be further processed to result in a fecal extract. Without intending to be limited by theory, it is possible the homogenization step causes the fecal sample to pass through the filter multiple times.

The linear filter can be described by sieve size, also referred to as pore size, in millimeters (mm) or sieve size based on the US Standard sieve sizes. The sieve size of the linear filter can be no greater than 2 mm, no greater than 1 mm, no greater than 0.7 mm, no greater than 0.65 mm, no greater than 0.6 mm, no greater than 0.55 mm, no greater than 0.5 mm, no greater than 0.33 mm, no greater than 0.25 mm, no greater than 0.212 mm, no greater than 0.18 mm, no greater than 0.15 mm, no greater than 0.125 mm, no greater than 0.106 mm, no greater than 0.09 mm, no greater than 0.075 mm, no greater than 0.063 mm, no greater than 0.053 mm, no greater than 0.045 mm, no greater than 0.038 mm, no greater than 0. 032 mm, no greater than 0.025 mm, no greater than 0.02 mm, or no greater than 0.01 mm. The sieve size of one or more filters can be no less than 0.212 mm, no less than 0.25 mm, no less than 0.3 mm, no less than 0.400 mm, no less than 0.5 mm, no less than 0.55 mm, no less than 0.6 mm, no less than 0.65 mm, no less than 0.7 mm, or no less than 0. mm. Sieve size of one or more of the filters can be, when described as US Standard sieve sizes, no greater than 400, no greater than 325, no greater than 270, no greater than 230, no greater than 200, no greater than 170, no greater than 140, no greater than 120, no greater than 100, no greater than 80, no greater than 70, no greater than 60, no greater than 35, no greater than 18, or no greater than 10. In one embodiment, the pores can be distributed at 200 to 300 pores per cm².

A cell extraction buffer is added to the fecal sample. The inventors observed that the use of gelatin in combination with ammonium phosphate aided in the release of biological material from the non-living material present in a fecal sample, accordingly, in some embodiments, a cell extraction buffer includes gelatin, ammonium phosphate, or both gelatin and ammonium phosphate.

A cell extraction buffer can be added to the fecal sample. In one embodiment a cell extraction buffer includes, but is not limited to a buffer, or both a buffer and a gelatin. In one embodiment, 1 part donor feces may be combined with 5 parts cell extraction buffer (e.g., 50 grams of donor feces may be combined with 250 ml cell extraction buffer) and homogenized. An example of a buffer includes, but is not limited to, (NH₄)₂HPO₄. The buffer, for instance, (NH₄)₂HPO₄, can be present at a concentration of at least 0.01 M, at least 0.05 M, or at least 0.1 M, and at a concentration of no greater than 1 M, no greater than 0.5 M, or no greater than 0.1 M. Examples of ranges of a buffer include, but are not limited to, 0.01 M to 1 M, and 0.05 M to 0.5 M. The gelatin is typically a mixture of peptides and proteins commonly derived from collagen. In one embodiment, the gelatin can be partially hydrolyzed. In one embodiment, the gelatin can be USP grade. The gelatin can be present at a concentration of at least 0.01% volume/volume (v/v), at least 0.05% v/v, or at least 0.1% v/v, and at a concentration of no greater than 1% v/v, no greater than 0.5% v/v, or no greater than 0.1% v/v. In one embodiment, a 10% v/v gelatin solution in water is adjusted to pH 10.0 and is partially hydrolyzed by autoclaving for 10 min at 15 PSI (Kingsley and Bohlool, Appl Environ Microbiol. 1981 August; 42 (2): 241-8. doi: 10.1128/aem.42.2.241-248.1981). Examples of ranges of gelatin in a cell extraction buffer include, but are not limited to, 0.01% v/v to 1% v/v, and 0.05% v/v to 0.5% v/v.

A cell extraction buffer can also include other components, including but not limited to a nonionic surfactant and emulsifier. Examples of a nonionic surfactant and emulsifier include, but are not limited to, polysorbate 80 (available under the trade name TWEEN-80®), Tween 60, and Nonident P40. A polysorbate 80 can be present at a concentration of at least 0.001%, at least 0.005%, or at least 0.01%, and at a concentration of no greater than 0.1%, no greater than 0.05%, or no greater than 0.01%.

The fecal sample in the container is subjected to conditions that homogenize the sample. In one embodiment, such conditions can be obtained through use of a paddle blender, also known in the art as a stomacher. Typically, the sample is homogenized until it is a slurry. Force can then be applied to the slurry to push the liquid component and biological material through the linear filter while preventing passage of the non-living material. The liquid component containing biological material is removed and the non-living material (the filtered material) resuspended in a diluent, such as a saline solution. Saline containing solutions useful in maintaining the viability of biological material present in a fecal sample are known, and in one embodiment includes phosphate buffered saline at pH 7.0. This can be subjected to conditions that homogenize the sample, and force applied to the resulting slurry to push the liquid component and biological material through the linear filter while preventing passage of the non-living material. The second liquid component containing biological material is removed and combined in a container suitable for centrifugation. The container can be purged with nitrogen gas to maintain conditions for anaerobes. The force of a centrifugation step can be at any speed that causes the separation of biological material from liquid but does not cause loss of therapeutic efficacy of the composition in MTT. Care should be taken as damage of the fecal microbes can be increased at greater speeds, and the fecal microbes can be more difficult to resuspend. Examples of suitable conditions include speeds resulting in at least 3,000×g, at least 4,000×g, or at least 5,000×g. Other examples of suitable conditions include speeds resulting in no greater than 9,000×g, no greater than 8,000×g, or no greater than 7,000×g. Suitable ranges include at least 3,000×g to no greater than 9,000×g, at least 4,000×g to no greater than 8,000×g, and at least 5,000×g to no greater than 7,000×g. Typically, the volume of diluent added back to the biological material after removal of the supernatant is reduced to cause concentration of the material. For instance, the diluent added back to the material can be one half the volume present before the separation step. The washing procedure of centrifugation followed by removal of the supernatant and resuspending the pellet in a reduced volume of diluent is repeated at least twice for a total of four washes.

The final pellet resulting from either the pressurized filter press method or the linear filtration method is resuspended in a diluent. The diluent used can be a volume that is no greater than 50% of the starting volume (e.g., the volume present when the fecal sample and cell extraction buffer are combined), no greater than 40% of the starting volume, no greater than 30% of the starting volume, no greater than 20% of the starting volume, or no greater than 10% of the starting volume. The final volume is typically dictated by the final numbers of bacteria desired after processing as described herein (e.g., adding a cryoprotectant or freeze drying). Typically, resuspending the final pellet in an aqueous composition results in a final slurry. The final slurry can be referred to herein as a fecal extract.

Cell numbers in a fecal extract can be determined. Methods for determining cell numbers include any combination of the Bacteria Counting Kit (ThermoFisher, Waltham, MA), an epifluorescence microscope, equipped with a 470-490 nm excitation filter and 500 nm cut-on long pass emission filter, and a Petroff Hauser counting chamber. A membrane integrity assay can be used to directly enumerate microbes present in a diluted sample and to calculate the percentage of microbes with intact membranes, thereby indicating potential viability. An example of a membrane assay is the BacLight live/dead membrane integrity assay (ThermoFisher, Waltham, MA) and a microscope and counting chamber.

In one embodiment, a composition described herein of fecal microbes is freeze-dried to form solid dried powder. A composition of fecal microbes is mixed with a cryoprotectant and/or lyoprotectant and subjected to conditions that result in freeze-drying. In those embodiments where the freeze-dried composition will be used to make a liquid solution for oral consumption, a composition of fecal microbes can be mixed with a flavor agent, and in other embodiments a flavor agent is added when the freeze-dried composition is reconstituted. Conditions that result in freeze-drying typically include freezing the sample, and reducing the pressure surrounding the frozen sample to remove water from the sample. Once freeze-dried, the composition may be further processed by subjecting the dried material to force sufficient to break up the material into a powder that can be easily stored until used. In one embodiment, the powder may be used to form granules, compressed tablets, pills, capsules, wafers, and the like. In one embodiment, the freeze-dried material can be formulated such that it is released from a capsule into the small or large intestine or the colon, and not the stomach.

In one embodiment, a freeze-dried composition described herein is reconstituted to result in a liquid suspension. A freeze-dried composition is mixed with an aqueous diluent. The freeze-dried composition can include a flavor agent and/or a gut-transit protectant, or a flavor agent and/or a gut-transit protectant can be added at the time of reconstitution. In one embodiment, the aqueous diluent includes a dairy product such yogurt or a milk product. Examples of milk include whole milk or milk having reduced fat, such as 1% or 2% milk fat, and the milk can be regular or chocolate milk, or vegetable-based milks. Typically, the reconstitution occurs using conditions that maintain the efficacy of the composition, e.g., conditions that reduce inactivating the microbes present. Appropriate conditions can include gentle mixing to dissolve or suspend the freeze-dried composition in the aqueous diluent.

The present disclosure is further directed to methods of using the freeze-dried compositions described herein. One method includes administering to a subject in need thereof an effective amount of a composition described herein. In one embodiment, the freeze-dried composition is reconstituted. The subject can be anyone in need thereof. In some embodiments, the subject is an individual that has had an intestinal surgery. Examples of intestinal surgery include, but are not limited to, those that can be used to treat diverticulitis, colorectal cancer such as colon adenocarcinoma, and inflammatory bowel disease such as Crohn's disease, ulcerative colitis, or polyps. In one embodiment, an intestinal surgery includes intestinal resection and reconnection (anastomosis). The anastomosis can be, but is not limited to, ileocolic anastomosis, colo-colonic anastomosis, coloanal anastomosis, or ileoanal anastomosis. In one embodiment, an anastomosis is not possible and a stoma is created. A stoma can be a temporary colostomy, a permanent colostomy, or an ileostomy. Examples of intestinal surgeries having intestinal resection and reconnection include, but are not limited to, sigmoid colectomy. A subject can be pediatric or adult. A subject may be a mammal, such as a human. In some embodiments animal models may be used, such as a mammal, including a rat, a mouse, a hamster, a gerbil, or a primate.

The administering is done under conditions suitable for deposition of the composition in a region of the large or small intestine such that the biological material in the composition colonizes the small intestine and/or colon. For instance, administration may be into an upper gastrointestinal tract, as well as a lower gastrointestinal tract, e.g., the terminal ileum, cecum, colonic areas containing diverticulosis, and rectum. In one embodiment the administering may be oral, such as by a capsule or a tablet. In one embodiment the administering may be by intubation, such as by nasogastric tube or post-pyloric nasojejunal feeding tube, of a freeze-dried composition that has been reconstituted. In one embodiment the administering may be rectal, for instance by a colonoscope, enema, or suppository. Conditions that are “suitable” for an event to occur, or “suitable” conditions are conditions that do not prevent such events from occurring. Thus, these conditions permit, enhance, facilitate, and/or are conducive to the event. As used herein, an “effective amount” relates to a sufficient amount of a composition described herein, to provide the desired effect.

In some embodiments, an effective amount is an amount that is sufficient to affect a reduction of a post-operative complication such as delayed return of bowel function (ROBF), infection (including anastomotic leaks (AL) and surgical site infections (SSI)), hospital length of stay, morbidity, and/or mortality. A reduction in a post-operative complication is, for instance, at least 10%, at least 20%, at least 30%, at least 40%, at least 50%, at least 60%, at least 70%, at least 80%, at least 90%, or at least 100% in a measured post-operative complication as compared to a control, a non-treated subject, or the subject prior to administration of the composition. For instance, in one embodiment an “effective amount” is an amount effective to alter the relative abundance of certain prokaryotic cells, or the concentration of certain SCFAs, or the concentration of certain BCFAs, or any combination thereof.

The relative abundance of certain prokaryotic cells in the gut of a subject is often increased on the day of surgery and after surgery in comparison to the subject before the surgery. In one embodiment, the relative abundance of one or more members of the family Streptococcaceae, such as one or more members of the genus Streptococcus is increased in a subject on the day of surgery and after surgery in comparison to the subject before the surgery. In one embodiment, the relative abundance of one or more members of the family Enterococcaceae, such as one or more members of the genus Enterococcus, is increased in a subject on the day of surgery and after surgery in comparison to the subject before the surgery. The presence of these prokaryotic cells is highly undesirable, as members of these bacterial taxa produce enzymes that degrade collagen, which contribute to the development of anastomotic leaks (Christley S, et al., PLOS One 15 (4): e0232165, 2020; Hernandez-Gonzalez P I, et al., Colorectal Dis, 25:2187-2197, 2023; Shogan B D, et al., Sci Transl Med, 2015 May 6; 7 (286): 286ra68. doi: 10.1126/scitranslmed.3010658). The methods disclosed herein include reducing, in a subject that has had an intestinal surgery, the relative abundance of one or more members of the family Streptococcaceae, such as one or more members of the genus Streptococcus, and/or reducing the relative abundance of one or more members of the family Enterococcaceae, such as one or more members of the genus Enterococcus, in the colon of a subject. The relative abundance in a recipient's colon after an administration can be decreased by at least 1%, at least 5%, at least 10%, at least 15%, at least 20%, at least 25%, at least 30%, at least 35%, at least 40%, at least 45%, or at least 50%, compared to the recipient's colon before the administration. Methods for determining the relative abundance of prokaryotic cells in a stool sample are known in the art.

The relative abundance of certain health-associated commensal prokaryotic cells in the gut of a subject is often decreased on the day of surgery and after surgery in comparison to the subject before the surgery. In one embodiment, the relative abundance of one or more members of the family Bacteroidaceae, such as one or more members of the genus Bacteroides, is decreased in a subject on the day of surgery and after surgery in comparison to the subject before the surgery. In one embodiment, the relative abundance of one or more members of the family Lachnospiraceae, including members of the genera Blautia, Coprococcus, Dorea, Lachnospira, Oribacterium, and Roseburia, and/or the family Ruminococcaceae (also known as the family Oscillospiraceae), such as one or more members of the genera Ruminococcus and/or Faecalibacterium is decreased in a subject on the day of surgery and after surgery in comparison to the subject before the surgery. The methods disclosed herein can include increasing, in a subject that has had an intestinal surgery, the relative abundance of one or more members of the family Bacteroidaceae, such as one or more members of the genus Bacteroides, and/or increasing the relative abundance of one or more members of the family Lachnospiraceae, including members of the genera Blautia, Coprococcus, Dorea, Lachnospira, Oribacterium, and Roseburia, and/or the family Ruminococcaceae, such as one or more members of the genera Ruminococcus and/or Faecalibacterium, in the colon of a subject. The relative abundance in a recipient's colon after an administration can be increased by at least 1%, at least 5%, at least 10%, at least 15%, at least 20%, at least 25%, at least 30%, at least 35%, at least 40%, at least 45%, or at least 50%, compared to the recipient's colon before the administration.

The methods disclosed herein can include increasing, in a subject that has had an intestinal surgery, the relative abundance of one or more members of the family Akkermansiaceae, including one or more members of the genus Akkermansia, such as, but are not limited to, Akkermansia mucinophila, and/or increasing the relative abundance of one of more members of the family Coriobacteriaceae include, but are not limited to, one or more members of the genus Collinsella, in the colon of a subject. The relative abundance in a recipient's colon after an administration can be increased by at least 1%, at least 5%, at least 10%, at least 15%, at least 20%, at least 25%, at least 30%, at least 35%, at least 40%, at least 45%, or at least 50%, compared to the recipient's colon before the administration.

The amount of short-chain fatty acids (SCFAs) in the gut of a subject is often decreased on the day of surgery and after surgery in comparison to the subject before the surgery. Examples of SCFAs include, but are not limited to, acetic acid, propionic acid, butyric acid, valeric acid, 2-ethylbutyric acid, and 4-methylvaleric acid. In one embodiment, the SCFA is butyric acid. In one embodiment, the concentration of an SCFA is decreased in a subject on the day of surgery and after surgery in comparison to the subject before the surgery. The methods disclosed herein include increasing, in a subject that has had an intestinal surgery, the concentration of one or more SCFAs in the colon of a subject. The concentration of one or more SCFA in a recipient's colon after an administration can be increased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the recipient's colon before the administration. Methods for determining the concentration of a SCFA in a stool sample are known in the art.

The amount of branched-chain fatty acids (BCFAs) in the gut of a subject is often decreased on the day of surgery and after surgery in comparison to the subject before the surgery. Examples of BCFAs include, but are not limited to, 2-methylbutyrate, isobutyrate, and isovalerate. In one embodiment, the concentration of a BCFA is decreased in a subject on the day of surgery and after surgery in comparison to the subject before the surgery. The methods disclosed herein include increasing, in a subject that has had an intestinal surgery, the concentration of one or more BCFAs in the colon of a subject. The concentration of one or more BCFA in a recipient's colon after an administration can be increased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the recipient's colon before the administration. Methods for determining the concentration of a BCFA in a stool sample are known in the art.

The change in the relative abundance of one or more prokaryotic cell and/or concentration of one or more SCFA and/or concentration of one or more BCFA may be determined at, for instance, 1 day, 3 days, 10 days, 14 days, 30 days, 90 days, or 180 days after the administration of fecal material.

In one embodiment, an effective amount is an amount sufficient to result in at least 5×10¹⁰ cells, at least 5.5×10¹⁰ cells, at least 6×10¹⁰ cells, at least 6.5×10¹⁰ cells, at least 7×10¹⁰ cells, at least 7.5×10¹⁰ cells, at least 8×10¹⁰ cells, at least 8.5×10¹⁰ cells, at least 9×10¹⁰ cells, at least 9.5×10¹⁰ cells, at least 1×10¹¹ cells, at least 2.5×10¹¹ cells, at least 3×10¹¹ cells, at least 3.5×10¹¹ cells, at least 4×10¹¹ cells, at least 4.5×10¹¹ cells, at least 5×10¹¹ cells, or at least 5.5×10¹¹ cells, at least 6×10¹¹ cells, at least 6.5×10¹¹ cells, at least 7×10¹¹ cells, at least 7.5×10¹¹ cells, at least 8×10¹¹ cells, at least 8.5×10¹¹ cells, at least 9×10¹¹ cells, at least 9.5×10¹¹ cells, at least 1×10¹² cells, at least 1.5×10¹² cells, at least 2×10¹² cells, at least 2.5×10¹² cells, at least 3×10¹² cells, at least 3.5×10¹² cells, at least 4×10¹² cells, at least 4.5×10¹² cells, at least 5×10¹² cells, or at least 5.5×10¹² cells, at least 6×10¹² cells, at least 6.5×10¹² cells, at least 7×10¹² cells, at least 7.5×10¹² cells, or at least 8×10¹² cells administered to the subject. In one embodiment, an effective amount is an amount sufficient to result in 5×10¹⁰ to 8×10¹² cells delivered to the colon, or 1×10¹¹ to 3×10¹² cells administered to the subject.

In one embodiment, an effective amount is an amount sufficient to result in at least 5×10¹⁰ cells, at least 5.5×10¹⁰ cells, at least 6×10¹⁰ cells, at least 6.5×10¹⁰ cells, at least 7×10¹⁰ cells, at least 7.5×10¹⁰ cells, at least 8×10¹⁰ cells, at least 8.5×10¹⁰ cells, at least 9×10¹⁰ cells, at least 9.5×10¹⁰ cells, at least 1×10¹¹ cells, at least 2.5×10¹¹ cells, at least 3×10¹¹ cells, at least 3.5×10¹¹ cells, at least 4×10¹¹ cells, at least 4.5×10¹¹ cells, at least 5×10¹¹ cells, or at least 5.5×10¹¹ cells, at least 6×10¹¹ cells, at least 6.5×10¹¹ cells, at least 7×10¹¹ cells, at least 7.5×10¹¹ cells, at least 8×10¹¹ cells, at least 8.5×10¹¹ cells, at least 9×10¹¹ cells, at least 9.5×10¹¹ cells, at least 1×10¹² cells, at least 1.5×10¹² cells, at least 2×10¹² cells, at least 2.5×10¹² cells, at least 3×10¹² cells, at least 3.5×10¹² cells, at least 4×10¹² cells, at least 4.5×10¹² cells, at least 5×10¹² cells, or at least 5.5×10¹² cells, at least 6×10¹² cells, at least 6.5×10¹² cells, at least 7×10¹² cells, at least 7.5×10¹² cells, or at least 8×10¹² cells delivered to the colon. In one embodiment, an effective amount is an amount sufficient to result in 5×10¹⁰ to 8×10¹² cells delivered to the colon, or 1×10¹¹ to 3×10¹² cells delivered to the colon.

In some embodiments, an initial high dosage is followed by one or more lower maintenance doses. An initial high dosage can include an amount sufficient to result in at least 5×10¹⁰ cells, at least 5.5×10¹⁰ cells, at least 6×10¹⁰ cells, at least 6.5×10¹⁰ cells, at least 7×10¹⁰ cells, at least 7.5×10¹⁰ cells, at least 8×10¹⁰ cells, at least 8.5×10¹⁰ cells, at least 9×10¹⁰ cells, at least 9.5×10¹⁰ cells, at least 1×10¹¹ cells, at least 2.5×10¹¹ cells, at least 3×10¹¹ cells, at least 3.5×10¹¹ cells, at least 4×10¹¹ cells, at least 4.5×10¹¹ cells, at least 5×10¹¹ cells, or at least 5.5×10¹¹ cells, at least 6×10¹¹ cells, at least 6.5×10¹¹ cells, at least 7×10¹¹ cells, at least 7.5×10¹¹ cells, at least 8×10¹¹ cells, at least 8.5×10¹¹ cells, at least 9×10¹¹ cells, at least 9.5×10¹¹ cells, at least 1×10¹² cells, at least 1.5×10¹² cells, at least 2×10¹² cells, at least 2.5×10¹² cells, at least 3×10¹² cells, at least 3.5×10¹² cells, at least 4×10¹² cells, at least 4.5×10¹² cells, at least 5×10¹² cells, or at least 5.5×10¹² cells, at least 6×10¹² cells, at least 6.5×10¹² cells, at least 7×10¹² cells, at least 7.5×10¹² cells, or at least 8×10¹² cells administered to the subject, or an amount sufficient to result in at least 5×10¹⁰ cells, at least 5.5×10¹⁰ cells, at least 6×10¹⁰ cells, at least 6.5×10¹⁰ cells, at least 7×10¹⁰ cells, at least 7.5×10¹⁰ cells, at least 8×10¹⁰ cells, at least 8.5×10¹⁰ cells, at least 9×10¹⁰ cells, at least 9.5×10¹⁰ cells, at least 1×10¹¹ cells, at least 2.5×10¹¹ cells, at least 3×10¹¹ cells, at least 3.5×10¹¹ cells, at least 4×10¹¹ cells, at least 4.5×10¹¹ cells, at least 5×10¹¹ cells, or at least 5.5×10¹¹ cells, at least 6×10¹¹ cells, at least 6.5×10¹¹ cells, at least 7×10¹¹ cells, at least 7.5×10¹¹ cells, at least 8×10¹¹ cells, at least 8.5×10¹¹ cells, at least 9×10¹¹ cells, at least 9.5×10¹¹ cells, at least 1×10¹² cells, at least 1.5×10¹² cells, at least 2×10¹² cells, at least 2.5×10¹² cells, at least 3×10¹² cells, at least 3.5×10¹² cells, at least 4×10¹² cells, at least 4.5×10¹² cells, at least 5×10¹² cells, or at least 5.5×10¹² cells, at least 6×10¹² cells, at least 6.5×10¹² cells, at least 7×10¹² cells, at least 7.5×10¹² cells, or at least 8×10¹² cells delivered to the colon. It will be understood, however, that the total dosage of the compositions as disclosed herein will be decided by the attending physician within the scope of sound medical judgment. The exact amount required will vary depending on factors such as the type and extent of condition being treated, such as the type and extent of intestinal surgery.

Clearance of a subject's microbiota before the surgery is often helpful. Methods for clearance of existing microbiota are known and routine. In one example, clearance can be accomplished by mechanical bowel preparation (MBP), optionally with oral antibiotics and optionally with perioperative IV antibiotics. In one embodiment, clearance is accomplished by MBP with oral antibiotics and perioperative IV antibiotics. Examples of useful cocktail oral antibiotics include neomycin plus metronidazole. Perioperative IV antibiotics can be initiated 1-2 hours before incision, and can include cefazolin plus metronidazole, or clindamycin plus gentamicin.

Administration of the composition having the fecal material can begin when the antibiotics are expected to be at or near elimination from the subject. In one embodiment, the composition can be administered at least 12 hours, at least 24 hours, at least 36 hours, at least 48 hours, at least 52 hours, or at least 56 hours after the last antibiotic was administered. In one embodiment, the composition can be administered at post-operation day (POD) 1, POD2, or POD3. In one embodiment, the composition can be administered within 12 hours, within 24 hours, within 36 hours, within 48 hours, within 60 hours, or within 72 hours of the end of the intestinal surgery.

Terms used herein will be understood to take on their ordinary meaning in the relevant art unless specified otherwise. Several terms used herein and their meanings are set forth below.

As used herein, “relative abundance” refers to the number of members of a phylum, class, order, family, genus, or species compared to the number of members of all other taxa in a recipient's colon or stool. Such a comparison can be expressed as a percent. For instance, the number of members of a phylum, class, order, family, genus, or species in a recipient's colon after an administration can be increased or decreased by at least 1%, at least 5%, at least 10%, at least 15%, at least 20%, at least 25%, at least 30%, at least 35%, at least 40%, at least 45%, or at least 50%, compared to the recipient's colon before the administration.

As used herein, a short-chain fatty acid (SCFA) refers to fatty acids of two to six carbon atoms. Examples of SCFAs include, but are not limited to, acetic acid, propionic acid, butyric acid, valeric acid, 2-ethylbutyric acid, and 4-methylvaleric acid.

As used herein, a branched-chain fatty acid (BCFA) refers to fatty acids of two to six carbon atoms that include one or more methyl branches on the carbon chain. Examples of BCFAs include, but are not limited to, 2-methylbutyrate, isobutyrate, and isovalerate.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The invention is defined in the claims. However, below there is provided a non-exhaustive listing of non-limiting exemplary aspects. Any one or more of the features of these aspects may be combined with any one or more features of another example, embodiment, or aspect described herein.

### EXEMPLARY ASPECTS

Aspect 1. A method of decreasing the relative abundance of a member of the genus Streptococcus, a member of the genus Enterococcus, or a member of the genus Streptococcus and a member of the genus Enterococcus, in a patient that has undergone an intestinal surgery, the method comprising: administering to the patient an effective amount of a composition comprising a fecal extract and a pharmaceutically acceptable carrier, wherein the relative abundance of a member of the genus Streptococcus, a member of the genus Enterococcus, or a member of the genus Streptococcus and a member of the genus Enterococcus is reduced following administration of the composition.

Aspect 2. A method of increasing the relative abundance of one or more members of the family Bacteroidaceae, such as one or more members of the genus Bacteroides, and/or one or more members of the family Ruminococcaceae, and/or one or more members of the family Lachnospiraceae, and/or one or more members of the family Akkermansiaceae, and/or one or more members of the family Coriobacteriaceae, or any combination thereof in a patient that has undergone an intestinal surgery, the method comprising: administering to the patient an effective amount of a composition comprising a fecal extract and a pharmaceutically acceptable carrier, wherein the relative abundance of one or more members of the family Bacteroidaceae, the family Lachnospiraceae, the family Ruminococcaceae, the family Akkermansiaceae, or the family Coriobacteriacea, is increased following administration of the composition.

Aspect 3. A method of increasing the presence of short-chain fatty acids (SCFAs), the presence of branched-chain fatty acids (BCFAs), or both SCFAs and BCFAs in a patient that has undergone an intestinal surgery, the method comprising: administering to the patient an effective amount of a composition comprising a fecal extract and a pharmaceutically acceptable carrier, wherein the concentration of SCFAs and/or BCFAs is increased following administration of the composition.

Aspect 4. The method of any of aspects 1-3 or 4-19, wherein the composition comprises a reconstituted freeze-dried composition comprising biological material, a cryoprotectant or lyoprotectant or both cryoprotectant and lyoprotectant, wherein the freeze-dried composition is friable, and wherein the biological material comprises human gut, colon, or intestinal fecal microbes, and optionally the biological material comprises human gut, colon or intestinal bacteria.

Aspect 5. The method of any of aspects 1~4 or 6-19, wherein the lyoprotectant comprises trehalose.

Aspect 6. The method of any of aspects 1-5 or 7-19, wherein the composition further comprises a gut-transit protectant.

Aspect 7. The method of any of aspects 1-6 or 8-19, wherein the composition comprises no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% weight non-living material/weight biological material.

Aspect 8. The method of any of aspects 1-7 or 9-19, wherein the composition comprises 1×10¹⁰ to 5×10¹² cells.

Aspect 9. The method of any of aspects 1-8 or 10-19, wherein the relative abundance of Streptococcus, Enterococcus, or both Streptococcus and Enterococcus is reduced by at least 1%, at least 5%, or at least 10% following administration of the composition.

Aspect 10. The method of any of aspects 1-9 or 11-19, wherein the relative abundance of one or more members of the family Bacteroidaceae, one or more members of the family Lachnospiraceae, one or more members of the family Ruminococcaceae, one or more members of the family Akkermansiaceae, one or more members of the family Coriobacteriaceae, or any combination thereof, is increased by at least 1%, at least 5%, or at least 10% following administration of the composition.

Aspect 11. The method of any of aspects 1-10 or 12-19, wherein the one or more members of the family Bacteroidaceae, comprise one or more members of the genus Bacteroides, one or more members of the family Lachnospiraceae comprises one or more members of the genera Blautia, Coprococcus, Dorea, Lachnospira, Oribacterium, and Roseburia, one or more members of the family Ruminococcaceae comprises one or more members of the genera Ruminococcus and Faecalibacterium, one or more members of the family Akkermansiaceae comprises one or more members of the genus Akkermansia, and one or more members of the family Coriobacteriaceae comprises one or more members of the genus Collinsella.

Aspect 12. The method of any of aspects 1-11 or 13-19, wherein the concentration of SCFAs is increased by at least 1%, at least 5%, or at least 10% following administration of the composition.

Aspect 13. The method of any of aspects 1-12 or 14-19, wherein the composition is administered within 12 hours, within 24 hours, within 36 hours, within 48 hours, within 60 hours, or within 72 hours of the end of the intestinal surgery.

Aspect 14. The method of any of aspects 1-13 or 15-19, wherein the administering is by oral administration.

Aspect 15. The method of any of aspects 1-14 or 16-19, wherein the administering is by intubation.

Aspect 16. The method of any of aspects 1-15 or 17-19, wherein the intubation is by post-pyloric nasojejunal feeding tube.

Aspect 17. The method of any of aspects 1-16 or 18-19, wherein the relative abundance is determined 1 day, 3 days, 10 days, 14 days, 30 days, 90 days, or 180 days after the administration of fecal material.

Aspect 18. The method of any of aspects 1-17 or 19, wherein the concentration of one or more SCFA or BCFA is determined 1 day, 3 days, 10 days, 14 days, 30 days, 90 days, or 180 days after the administration of fecal material.

Aspect 19. The method of any of aspects 1-18, wherein the relative abundance of a microbe or the concentration of SCFAs or BCFAs is the relative abundance or the concentration in the colon of the patient.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

### Microbiota Transplant Therapy Reveals a Coordinated Postoperative Recovery Program Across Microbial, Metabolic, and Immune Systems

Postoperative complications such as ileus, surgical site infection, and anastomotic leak remain major drivers of morbidity, prolonged hospitalization, and healthcare costs following colorectal surgery, despite widespread adoption of Enhanced Recovery After Surgery protocols and standardized perioperative care. (1-3) Although traditionally viewed as distinct clinical entities, increasing evidence suggests that these complications share overlapping biological drivers rooted in disruption of the gut ecosystem and its downstream effects on inflammation, mucosal healing, and immune regulation. (4) Postoperative ileus, for example, is now understood as a neuroinflammatory condition characterized by impaired epithelial repair and altered gut-brain signaling, processes influenced by microbial metabolites and host-microbiota-immune interactions. (3, 5) Similarly, specific microbial taxa have been implicated in the pathogenesis of surgical site infections and anastomotic leaks, including Enterococcus faecalis, which promotes collagen degradation, and Streptococcus species, which are enriched in postoperative infections and disrupted gut communities. (6-8) Together, the inventors determined that these observations identify the gut microbiome and its interaction with host immune responses as central and potentially modifiable determinants of surgical recovery. More broadly, they suggested to the inventors that postoperative recovery reflects an active biological reconstitution process rather than a passive return of bowel function.

The gut microbiome plays a central role in intestinal healing, immune homeostasis, and gastrointestinal motility, in large part through the production of short-chain fatty acids (SCFAs) that support epithelial integrity, regulate innate and adaptive immune function, and influence enteric neuroimmune signaling. (6, 9-13) However, perioperative interventions are practiced for surgical safety, including mechanical bowel preparation and antibiotic prophylaxis, (14, 15) profoundly disrupt microbial community structure and result in near-complete suppression of microbial metabolic activity, including loss of fermentative capacity and SCFA production. (16-18) This postoperative dysbiosis is characterized by depletion of SCFA-producing taxa and expansion of opportunistic pathobionts such as Enterococcus and Streptococcus, patterns that have been associated with delayed gastrointestinal recovery, sustained inflammation, and impaired immune resolution. (6-8) Across human and animal studies, this perioperative ecological collapse has been linked to impaired epithelial healing, weakened colonization resistance, and disrupted neuroimmune signaling critical for gut motility and function. (6, 13, 16, 17, 19-25) Despite growing recognition of the microbiome's role in postoperative physiology, there are currently no standardized clinical strategies designed to actively restore gut microbial composition, metabolic function, and immune homeostasis after surgery.

Microbiota transplant therapy (MTT) has demonstrated the capacity to induce durable microbial engraftment and restore metabolic function in other settings of severe dysbiosis, most notably Clostridioides difficile infection. (26-33) Whether this approach can be leveraged to accelerate biological recovery in the immediate postoperative period, and how it reshapes systemic immune responses following surgical injury, remains unknown. Importantly, the inventors determined that restoration of gut microbial communities would be expected not only to reestablish fermentative metabolism, but also to modulate systemic immune responses that are tightly coupled to postoperative inflammation, tissue repair, and recovery, given the established role of microbiota-derived signals in immune programming and resolution. (4, 23)

In this Phase I clinical study, the inventors evaluated the feasibility and biological impact of administering MTT during the early postoperative period following colorectal surgery. MTT was delivered via nasojejunal feeding tube on postoperative day 2, timed to follow perioperative antibiotic exposure and exploit the microbiota-depleted gut as a receptive niche for engraftment. (27, 34) Using longitudinal multi-omic profiling, the results show that targeted microbial reconstitution facilitates a coordinated postoperative recovery program that is otherwise obscured by perioperative perturbation, characterized by durable microbial engraftment, accelerated restoration of fecal short-chain and branched-chain fatty acids, and time-dependent remodeling of innate and adaptive immune compartments. Together, these findings define postoperative recovery as a coupled microbial-metabolic-immune process and establish MTT as a mechanistically informative intervention to interrogate and rationally design microbiome-targeted strategies to improve surgical recovery.

### Materials and Methods

### Ethical Statement

All research conducted in this study complied with relevant ethical regulations. The protocol was approved by the Institutional Review Board (study ID 00019579) and the Cancer Protocol Review Committee (SURG-2023-29833) at the University of Minnesota. Written informed consent was obtained from all participants prior to enrollment. The trial was registered at ClinicalTrials.gov (NCT06370884). The United States Food and Drug Administration approved the investigational product under an Investigational New Drug application (IND 28152). The investigational microbiota product was dispensed through the University of Minnesota Microbiota Therapeutics Program (MTP). (66)

### Donor Material and Microbiota Product Overview

Donor material was supplied by the University of Minnesota Microbiota Therapeutics Program, which maintains a rigorously screened donor pool using established clinical and laboratory criteria. (66) Donors underwent comprehensive medical history review, physical examination, and serial laboratory testing to exclude infectious diseases, gastrointestinal disorders, metabolic and autoimmune conditions, and risk factors for colonization with multidrug-resistant organisms. Screening included serologic, molecular, and culture-based testing for enteric pathogens and antimicrobial-resistant organisms at defined intervals, consistent with prior standardized fecal microbiota preparation protocols. (66)

MTT product (MTP-101-LR) was manufactured under current Good Manufacturing Practice conditions and stored frozen until use. Each participant received microbiota derived from a single donor and a single manufacturing batch. The administered dose ranged from 5-10×10¹¹ bacterial cells per treatment.

### Donor Screening and Eligibility Criteria

Donors were recruited and screened through the University of Minnesota Microbiota Therapeutics Program using established clinical and laboratory criteria. (66) Screening included a structured medical history questionnaire, physical examination, and serial laboratory testing to exclude gastrointestinal disease, metabolic disorders, autoimmune conditions, malignancy, recent antibiotic exposure, high-risk travel, and behaviors associated with infectious disease transmission. Laboratory screening included serologic, molecular, and culture-based testing for enteric pathogens, blood-borne viruses, and multidrug-resistant organisms, including extended-spectrum beta-lactamase-producing Enterobacterales, carbapenem-resistant organisms, vancomycin-resistant Enterococcus, and methicillin-resistant Staphylococcus aureus, consistent with prior standardized fecal microbiota donor screening protocols. (66)

### Microbiota Product Manufacturing and Quality Control

The investigational microbiota product was manufactured under current Good Manufacturing Practice conditions using previously described fecal microbiota processing workflows. (66) Donor stool was processed within a controlled laboratory environment using standardized homogenization, filtration, and cryopreservation procedures. Bacterial cell concentration was quantified using Petroff-Hausser chamber counting, and the final product was suspended in a cryoprotectant solution prior to aliquoting and storage at −80° C.

Lot release criteria included visual inspection, bacterial concentration thresholds, endotoxin testing, and sterility testing, consistent with prior fecal microbiota manufacturing standards. (66) Each participant received microbiota derived from a single donor and a single manufacturing lot.

### Clinical Study Design and MTT Administration

This single-center, open-label Phase I study evaluated the safety and feasibility of MTT in patients undergoing sigmoid colon resection with primary anastomosis for diverticulitis (n=9) or sigmoid colon adenocarcinoma (n=3) at the University of Minnesota Fairview Medical Center.

Eligible participants were adults aged 21-75 years scheduled for elective surgery. Exclusion criteria included inflammatory bowel disease, neutropenia (absolute neutrophil count <0.5×10⁹ cells/L), prior solid organ or bone marrow transplantation, current immunosuppressive therapy, active chemotherapy or radiation, ostomy creation, pregnancy, or limited life expectancy. This was an open-label study, and no randomization or blinding was performed. No formal power calculation was performed, as the study was designed to assess safety, feasibility, and mechanistic signals rather than clinical efficacy.

All patients received standardized mechanical bowel preparation with oral antibiotics and perioperative intravenous antibiotics per institutional protocol. Oral antibiotics included neomycin and metronidazole, and perioperative intravenous regimens were selected based on allergy status. Given known antibiotic elimination half-lives, (67) MTT was administered 48-52 hours after the final intravenous antibiotic dose, coinciding with near-complete antibiotic clearance in patients with normal renal function.

On the day of administration, the thawed microbiota suspension was delivered via a post-pyloric nasojejunal feeding tube, followed by saline flush. The feeding tube was subsequently removed, and postoperative diet advancement proceeded per routine clinical criteria.

### Sample Collection

Fecal samples were collected within two weeks prior to surgery (baseline), during the inpatient postoperative period, and on POD 14, 30, 90, and 180. Samples were stored at −20° C. following collection and transferred on ice for long-term storage at −80° C. until analysis.

Fecal samples were collected using sterile containers and immediately stored at −20° C. prior to transfer on ice to −80° C. storage, consistent with established microbiome preservation practices. (69) Peripheral blood samples were collected in EDTA tubes and stabilized immediately using a whole-blood stabilizing reagent validated for downstream immune profiling. (84) All samples were de-identified and processed in batch to minimize technical variability.

Peripheral blood samples were collected at baseline and on POD 1, 2, 3, 14, 30, 90, and 180. Routine clinical laboratory testing was performed at each time point, and aliquots were reserved for immune profiling.

Fecal DNA Extraction and 16S rRNA Gene Sequencing

DNA was extracted from fecal samples using a standardized commercial kit optimized for inhibitor removal. The V4 hypervariable region of the 16S rRNA gene was amplified using the 515F/806R primer set (68) and sequenced using paired-end, dual-indexed reads at a read length of 300 nucleotides on the Illumina MiSeq or Element AVITI instruments, as previously described. (69) Negative extraction and amplification controls were included in all sequencing runs and did not yield detectable amplicons.

16S rRNA Gene Sequencing, Quality Control, and Source Attribution

Raw sequencing reads were processed using mothur (v.1.41) following a previously published pipeline. (72, 73) Briefly, paired-end reads were assembled, quality-filtered, and aligned to the SILVA reference database (ver. 138). (74) Sequences were subjected to a 2% preclustering step to reduce sequencing error, (75) screened for chimeras using UCHIME, (76) and classified taxonomically using the Ribosomal Database Project reference database (ver. 18). (78) Operational taxonomic units (OTUs) were assigned using the OptiClust algorithm at 99% similarity. (77)

A median of 25,043 (interquartile range 14,491-945,436) sequence reads per sample were obtained. For unbiased statistical comparisons, (96) samples were rarefied to 5,000 sequence reads by random subsampling, and samples with fewer sequence reads were removed from the data set. After rarefaction, the mean estimated Good's coverage was 98.7±1.3%, with a mean of 116±48 OTUs observed per sample.

Negative extraction and amplification controls were included in all sequencing runs and did not yield detectable amplicons. Samples failing sequencing depth or quality control thresholds were excluded from downstream analyses, consistent with established microbiome quality control standards. (73)

Donor similarity and source attribution were assessed using SourceTracker, a Bayesian framework that estimates the proportion of microbial communities derived from predefined source environments. (83) Donor and recipient baseline stool samples were designated as source communities, and recipient fecal samples after baseline were treated as sinks. Analyses were performed on rarefied genus-level abundance tables using default SourceTracker parameters. Posterior distributions were estimated using Gibbs sampling, and donor contribution proportions were summarized across posterior samples. SourceTracker outputs were used to estimate longitudinal donor similarity following MTT.

Alpha diversity was assessed using the Shannon index. (79) Beta diversity was calculated using Bray-Curtis dissimilarity (80) and visualized by principal coordinate analysis. (81) Community differences were evaluated using analysis of similarity (ANOSIM) with Bonferroni correction for multiple comparisons. (82) SourceTracker was used to estimate donor similarity using Bayesian inference. (83)

### Fecal Short-Chain and Branched-Chain Fatty Acid Quantification

Fecal short-chain and branched-chain fatty acids were quantified using a validated gas chromatography-mass spectrometry (GC-MS) protocol. (70, 71)

### Short-Chain and Branched-Chain Fatty Acid Quantification

Fecal short-chain (e.g., acetate, propionate, butyrate, valerate) and branched-chain (e.g., isobutyrate, isovalerate, and 2-methylbutyrate) fatty acids were quantified by gas chromatography-mass spectrometry using validated protocols. (70, 71) Quantified metabolites included acetate, propionate, butyrate, valerate, isobutyrate, isovalerate, and 2-methylbutyrate. Samples were homogenized, acidified, and extracted prior to analysis. Calibration curves were generated using authentic standards, and internal standards were included to control for extraction efficiency and instrument variability. Quality control samples were analyzed at regular intervals throughout each analytical run. Metabolite concentrations were normalized to wet stool weight, as previously described. (70, 71)

### Microbiome Bioinformatics and Statistical Analysis

Amplicon sequence data were processed using mothur (72) following a previously published pipeline. (73) Reads were quality-filtered, aligned to the SILVA reference database, (74) preclustered, (75) and screened for chimeras. (76) Operational taxonomic units were assigned using OptiClust, (77) and taxonomic classification was performed against the Ribosomal

### Immune Profiling and Feature Construction

Whole-blood samples were stabilized (84) immediately after collection and profiled by spectral flow cytometry using dedicated innate and adaptive antibody panels (35, 85). After standard preprocessing (quality filtering to remove debris/doublets/dead cells and arcsinh transformation), major lineages were identified by hierarchical gating and then subjected to unsupervised clustering using FlowSOM, with UMAP used for visualization of phenotypic structure. For each sample, immune features were defined as cluster frequencies (proportion of parent lineage) for downstream modeling. To focus on immune remodeling rather than baseline abundance, immune inputs were expressed as within-subject changes (A frequencies) across prespecified postoperative contrasts (innate: POD1→POD3; adaptive: POD1→POD30). Cluster solutions were reviewed using marker-expression heatmaps and stability across samples/batches prior to downstream analyses.

### Spectral Flow Cytometry Antibody Panels and Data Acquisition

Custom antibody panels were designed to profile innate (Table 1) and adaptive (Table 2) immune populations using spectral flow cytometry, following established best practices for high-dimensional immune phenotyping. (35, 85) Panel design was optimized using Cytek similarity and complexity indices to minimize spectral overlap and signal spread.

TABLE 1
Innate Immune Spectral Flow Cytometry Antibody Panel

| Sl. No | Antibody | Fluorophore | Vendor | Catalog # | Clone |
|---|---|---|---|---|---|
| 1 | CD45 | B548 | Cytek | SKU R7-20292 | HI30 |
| 2 | CD56 | BV785 | BD Biosciences | 564058 | NCAM 16.2 |
| 3 | CD3 | PE-Fire640 | BioLegend | 344860 | SK7 |
| 4 | CD19 | BYG710 | Cytek | R7-20009 | HIB 19 |
| 5 | CD14 | PECy7 | BD Biosciences | 557742 | 9095611 |
| 6 | CD7 | BV480 | BD Biosciences | 566119 | M - T701 |
| 7 | CD16 | BV570 | BioLegend | 302036 | 3G8 |
| 8 | HLA-DR | R840 | Cytek | SKUR7-20294 | L243 |
| 9 | CD33 | PE | BD Biosciences | 555450 | 1165676 |
| 10 | CD11b | B515 | BD Biosciences | 564517 | ICRF 44 |
| 11 | CD20 | B675 | Cytek | R7-20195 | 2H7 |
| 12 | CD5 | BV510 | Bio legend | 364018 | 017F12 |
| 13 | CD141 | BV750 | BD Biosciences | 747244 | 1A4 |
| 14 | CD1c | BV650 | BD Biosciences | 569617 | F 10/21A3 |
| 15 | CD123 | PECy7 | BD Biosciences | 551065 | 8250742 |
| 16 | Live/Dead | ViaDye ™ Red | Cytek | SKUR7-60008 | — |
| | | Fixable | | | |
| | | Viability Dye | | | |
| 17 | CD54 | BUV496 | Bio legend | 353126 | B297158 |
| 18 | CD184 | BUV395 | BD Biosciences | 740265 | 2B11/CXCR4 |
| 19 | CD49d | BUV737 | BD Biosciences | 748702 | L25 |
| 20 | CD63 | PerCP-Cy5.5 | Bio legend | 353020 | 85C6 |
| 21 | CD15 | APC | Bio legend | 323008 | B421788 |
| 22 | OLFM-4 | AF488 | Novus biotechne | 06415AF488 | D185256 |
| 23 | CD66b | BV421 | Bio legend | 392916 | 412789 |
| 24 | CD62L | PE-Dazzle594 | Bio legend | 304842 | B418094 |
| 25 | CD177 | BUV615 | BD Biosciences | 751453 | 507-3600 |

Antibody panel used for spectral flow cytometric characterization of innate immune cell populations following MTT. This panel was designed to identify and quantify neutrophil, monocyte, dendritic cell, natural killer cell, and myeloid-derived suppressor cell (MDSC) subsets using marker combinations defining activation state, maturation, adhesion, migration, and immune-associated phenotypes. Fluorochrome selection and panel design were optimized for spectral uniqueness and minimal signal spread using Cytek similarity and complexity indices. All samples were acquired on a five-laser Cytek Aurora system. Antibody vendor, catalog number, clone, and fluorophore information are provided.

TABLE 2
Adaptive Immune Spectral Flow Cytometry Antibody Panel

| Sl. No. | Antibody | Fluorophore | Vendor | Catalog # | Clone |
|---|---|---|---|---|---|
| 1 | CD 45 | B 548 | Cytek | R7-2029 | H130 |
| 2 | CD 19 | PE Fire 640 | BD Horizon | 302274 | HIB 19 |
| 3 | CD 20 | BV570 | BioLegend | 302332 | 2H7 |
| 4 | CD 8 | BV 785 | BioLegend | 344740 | SK1 |
| 5 | CD 38 | R 720 | Cytek | R720061 | HIT2 |
| 6 | IgD | V450 | BD Horizon | 561309 | AI6-2 |
| 7 | TCR alpha/Beta | BV 510 | BioLegend | 306734 | IP26 |
| 8 | CD3 | PerCP | BD BioSciences | 552851 | SK7 |
| 9 | CD161 | V-605 | BD BioSciences | 748284 | HP3G10 |
| 10 | HLA DR | R840 | Cytek | R7-26293 | L243 |
| 11 | CD 45RA | R685 | BD BioSciences | 755990 | 5H9 |
| 12 | CD 28 | BV750 | BioLengend | 302970 | CD28.2 |
| 13 | CD 27 | BV650 | BioLegend | 302828 | 0323 |
| 14 | CD 4 | BB515 | BD Horizon | 566056 | L200 |
| 15 | CD 183 | R 659 | Cytek | RC-00049 | G025H7 |
| 16 | CD 185 | PerCP eFlour 710 | Invitrogen | 46918542 | MU5UBEE |
| 17 | CD 197 | BYG781 | Cytek | RC-00048 | G043H7 |
| 18 | CD25 | BYG575 | Cytek | RC-00044 | BC96 |
| 19 | CD194 | BV421 | BioLegend | 359414 | L291H4 |
| 20 | CD127 | BV 711 | BioLegend | 351328 | A019D5 |
| 21 | TCR Gamma/Delta | BYG710 | Tonbo | | |
| 22 | Live/Dead | ViaDye ™ | Cytek | SKUR7-60008 | — |
| | (ViaDye Red) | Red Fixable | | | |
| | | Viability Dye | | | |
| 23 | CD196 | RB705 | BD Biosciences | 566477 | 11A9 |

Antibody panel used for spectral flow cytometric characterization of adaptive immune cell populations following MTT. This panel enables identification of CD4⁺ and CD8⁺ T cell subsets, regulatory T cells, γδ T cells, and B cell populations across differentiation and activation states using established surface marker combinations. The panel was optimized for spectral performance using Cytek similarity and complexity indices to minimize signal overlap. All samples were acquired on a five-laser Cytek Aurora system. Antibody vendor, catalog number, clone, and fluorophore information are listed.

Data acquisition was performed on a five-laser Cytek Aurora system using standardized instrument settings. Spectral unmixing was performed using single-stain controls, and batch effects were assessed across acquisition days. (85)

### Immune Data Preprocessing and Quality Control

Flow cytometry data were compensated, transformed using an arcsinh transformation, and quality-filtered to remove debris, doublets, and dead cells, consistent with established immune cytometry workflows. (35) Cell populations were gated using a hierarchical strategy prior to high-dimensional analysis. Samples failing predefined quality control thresholds were excluded from downstream analyses.

### Unsupervised Immune Clustering and Dimensionality Reduction

High-dimensional immune data were analyzed using unsupervised clustering and dimensionality reduction approaches. Uniform Manifold Approximation and Projection (UMAP) was used for visualization of cellular phenotypes. (97) Clustering was performed using FlowSOM followed by metaclustering to identify phenotypically distinct cell populations. (98) Cluster identities were defined based on relative marker expression patterns rather than predefined functional labels, consistent with prior immune systems biology studies. (99)

Differential cluster abundance and marker expression were assessed using paired statistical tests with correction for multiple comparisons.

### Multi-Omics Integration and Latent Variable Modeling

To integrate microbiome composition and metabolite profiles, partial least squares regression was applied to genus-level microbial abundance data and fecal SCFA concentrations. (86-89) Data were centered and scaled prior to modeling. Latent axes were derived using stability selection to identify reproducible features contributing to shared variance across data types. (87, 88) Axis scores were calculated for each sample and used for downstream analyses.

### Shared-Embedding Correlation Network (SECN) Construction

Shared-embedding correlation networks were constructed by embedding microbiome, metabolite, and immune features into a low-dimensional shared space, as previously described. (90-93) Sparse correlation matrices were estimated using partial Spearman correlations with false discovery rate correction. Network sparsification was performed to retain robust feature associations.

Connector nodes were defined as features with statistically significant edges linking all three data layers. Enrichment of immune versus non-immune features among connector nodes was assessed using Fisher's exact test. (95)

### Lead-Lag Analysis

Time-ordered associations between immune features were assessed using baseline-adjusted lead-lag analysis, as previously described. (91, 92, 94) Partial Spearman correlations were computed between features at sequential timepoints while controlling for baseline values. Associations were retained based on effect size thresholds and false discovery rate correction.

### Multi-Omics Integration and Network Analyses

To integrate microbiome composition, fecal metabolite dynamics, and immune remodeling, we applied a two-stage multi-omics framework combining latent-factor modeling and shared-embedding network analysis. Partial least squares regression was used to derive shared latent axes linking genus-level microbiome structure with SCFA profiles. (86-89) Cross-omic coupling was further evaluated using a SECN framework based on low-rank embeddings and sparse correlation networks. (90-93)

Microbiome genus composition and fecal SCFA profiles were first integrated using partial least squares (PLS) to derive shared latent axes capturing maximal cross-omic covariance (e.g., Z2). Immune remodeling features (cluster-frequency A values) were then incorporated as a third data view in the shared-embedding correlation network framework, in which patient-aligned microbiome, SCFA, and immune-delta matrices were embedded into a low-rank shared space and used to construct sparse time-stratified cross-omic correlation networks. This framework enabled identification of phase-specific immune-(microbe, SCFA) coupling, baseline-adjusted lead-lag associations between innate and adaptive immune changes, and connector nodes bridging all three layers.

Time-ordered immune coordination was assessed using baseline-adjusted lead-lag analyses with partial Spearman correlations and multiple-testing correction. (91, 92, 94) Edges were retained if they satisfied predefined effect-size thresholds (|ρ|≥0.30), false discovery rate correction using the Benjamini-Hochberg procedure (q≤0.10), and sparsification criteria designed to limit network density and stabilize inference under small-n conditions. Connector-node analyses were performed to identify features bridging microbiome, metabolite, and immune layers, with enrichment testing performed using Fisher's exact test. (95)

### Statistical Analysis

Statistical analyses were performed using R (v.4.3). Paired comparisons were conducted using non-parametric tests where appropriate. Multiple-testing correction was applied using the Benjamini-Hochberg method. (92) Statistical significance was defined as adjusted P<0.05 unless otherwise specified.

### Results

### Study Cohorts

Twelve patients who consented to receive MTT were compared with 12 non-MTT controls drawn from a prior study who underwent identical sigmoid colectomy without MTT. (16, 17) Baseline demographic and perioperative characteristics were well balanced between groups (Table 3). All patients received a standardized mechanical bowel preparation with preoperative oral antibiotics, followed by perioperative intravenous antibiotics tailored to tolerance and allergy history.

TABLE 3
Patient Demographics and Clinical Characteristics.

| | MTT (n = 12) | Non-MTT Controls (n = 12) |
|---|---|---|
| Male sex, n (%) | 4 (33) | 8 (66) |
| Age, years | 54.0 ± 13.1 | 53.0 ± 11.3 |
| BMI, kg/m² | 28.2 ± 5.2 | 29.7 ± 4.4 |
| Preoperative albumin, g/dL | 4.3 ± 0.3 | 3.9 ± 0.3 |
| Charlson comorbidity index | 2.6 ± 1.4 | 1.4 ± 1.4 |
| Smoking status, n (%) | | |
| Active smoker | 1 (8) | 2 (17) |
| Former smoker | 6 (50) | 4 (33) |
| Non-smoker | 5 (42) | 6 (50) |
| Chronic narcotics, n (%) | 1 (8) | 1 (8) |
| History of diabetes mellitus, n (%) | 1 (8) | 2 (17) |
| Metformin | 1 (8) | 1 (8) |
| Preoperative bowel prep, n (%) | | |
| Polyethylene glycol and Magnesium citrate | 12 (100) | 12 (100) |
| Preoperative oral antibiotics, n (%) | | |
| Neomycin + metronidazole | 12 (100) | 12 (100) |
| Perioperative intravenous antibiotics, n (%) | | |
| Cefazolin+ metronidazole | 11 (92) | 3 (25) |
| Clindamycin + gentamicin | 1 (8) | 0 (0) |
| Ciprofloxacin + metronidazole | 0 (0) | 4 (33) |
| Cefotetan | 0 (0) | 2 (17) |
| Ertapenem | 0 (0) | 3 (25) |
| Disease pathology, n (%) | | |
| Sigmoid diverticulitis | 9 (75) | 9 (75) |
| Sigmoid colon cancer | 3 (25) | 3 (25) |
| Postoperative complications, n (%) | | |
| None | 10 (83) | 11 (92) |
| Anastomotic leak/sepsis | — | — |
| Urinary tract infection | — | — |
| Surgical site infection | — | — |
| Deep organ space infection | — | — |
| Pneumonia | — | — |
| Ileus requiring nasogastric tube | 1 (8) | 1 (8) |
| Anemia requiring transfusion | — | — |
| Acute renal failure | — | — |
| Deep venous thrombosis | — | — |
| Return of bowel function, days | 1.7 ± 0.8 | 2.2 ± 1.3 |
| 30-day hospital readmission, n (%) | 1 (8) | 0 (0) |

Perioperative IV antibiotic selection differed by allergy/tolerance.
Continuous variables are presented as mean ± standard deviation.
Categorical data are shown as counts and percentages.
MTT = Microbiota Transplant Therapy.

Postoperatively, one control patient developed a small bowel ileus requiring nasogastric decompression. One patient in the MTT group experienced a gastric ileus with preserved colonic function, which was similarly managed with nasogastric decompression and resolved with full recovery of gastric function. No adverse events were attributed to MTT, supporting the feasibility and safety of the intervention. Although not powered for clinical endpoints, MTT recipients demonstrated a numerical trend toward earlier return of bowel function compared with matched non-MTT controls (mean 1.7±0.8 vs 2.2±1.3 days; P=0.28).

### MTT Drives Durable Microbial Engraftment and Alters Community Structure

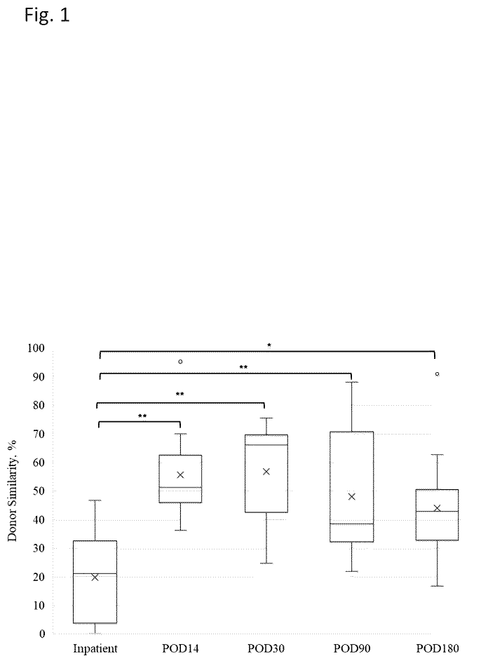

Prior to MTT, recipient gut microbiota shared limited similarity with the donor community (median 19.6%, IQR 7.7-30.6%). By postoperative day (POD) 14, donor similarity increased to 51.0% (IQR 44.1-58.2%; P<0.001) and remained significantly elevated at POD30 (51.4%, IQR 40.0-66.5%; P<0.001), POD90 (68.2%, IQR 37.1-83.5%; P<0.001), and POD180 (36.9%, IQR 17.6-56.9%; P<0.001) (FIG. 1), indicating durable microbial engraftment following MTT.

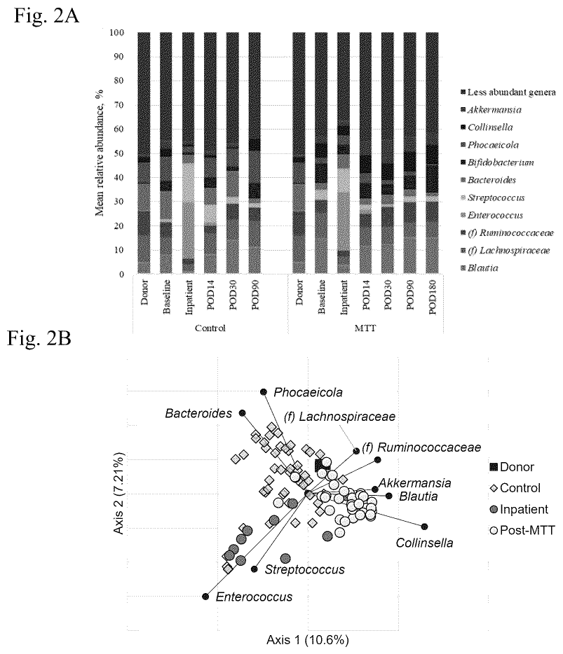

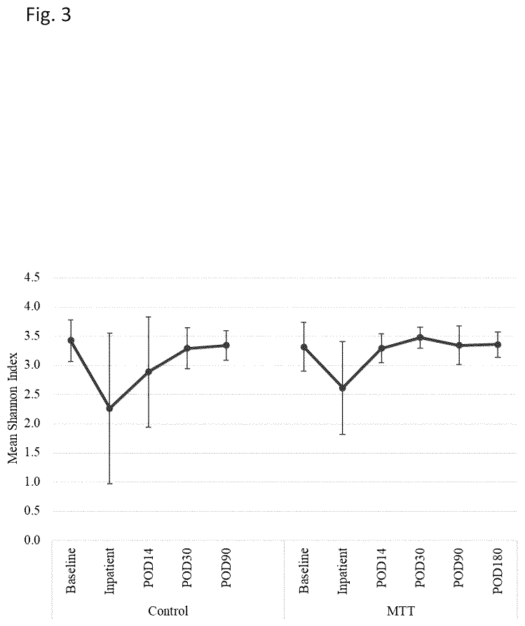

Consistent with these compositional shifts, beta-diversity analyses demonstrated sustained divergence in community composition between MTT recipients and non-MTT controls beginning at POD14 (FIGS. 2A-2B). Alpha diversity declined postoperatively in both groups; however, MTT recipients exhibited a trend toward higher alpha diversity at POD14, consistent with accelerated microbial recovery (FIG. 3).

### MTT Enriches Early-Recovery Taxa and Blunts Postoperative Pathobiont Expansion.

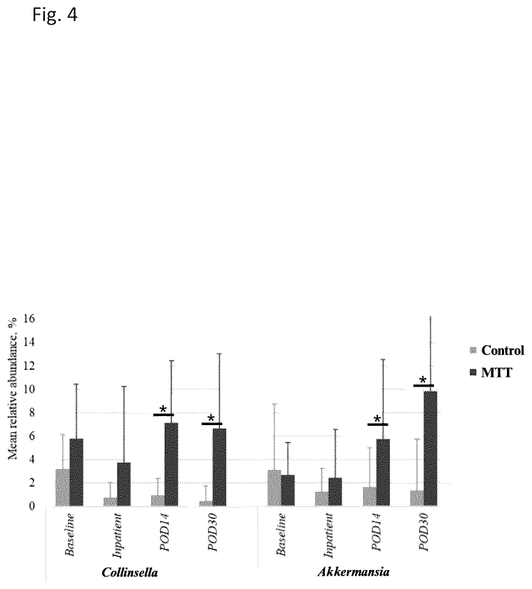

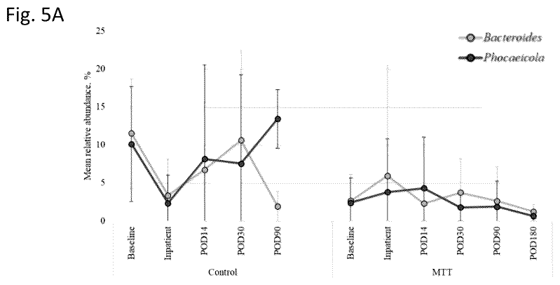

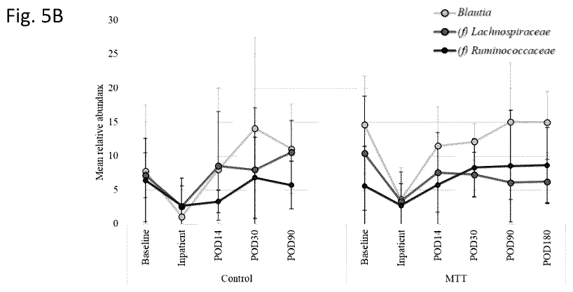

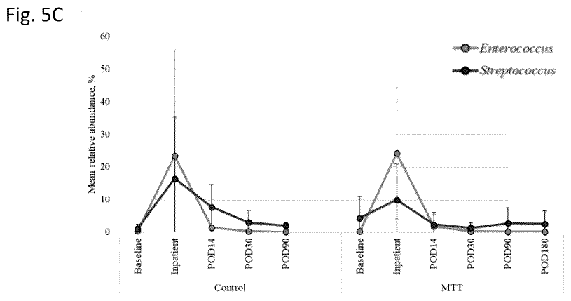

MTT was associated with distinct taxonomic shifts compared with non-MTT controls (FIG. 4). By POD14, MTT recipients exhibited significantly higher relative abundances of Akkermansia (P=0.018) and Collinsella (P=0.004), with both taxa remaining elevated at POD30 (P=0.003 and P=0.022, respectively). In contrast, relative abundances of Bacteroides and Phocaeicola were not affected by MTT (FIG. 5A).

Canonical butyrate-producing taxa, including Blautia, members of the Ruminococcaceae, and Lachnospiraceae (FIG. 5B), declined postoperatively in both groups. Although between-group differences at POD14 and POD30 did not reach statistical significance, relative abundances of these taxa consistently trended higher among MTT recipients (P≥0.070). Streptococcus, a taxon commonly associated with postoperative dysbiosis and reduced fecal SCFA levels, also trended lower in the MTT group at POD14 (P=0.070; FIG. 5C).

Collectively, these taxonomic patterns suggest that MTT favors early recovery of putative keystone taxa while attenuating postoperative expansion of pathobionts, thereby establishing a microbial community structure supportive of downstream functional recovery.

MTT Accelerates Recovery of Short-Chain and Branched-Chain Fatty Acids after Surgery.

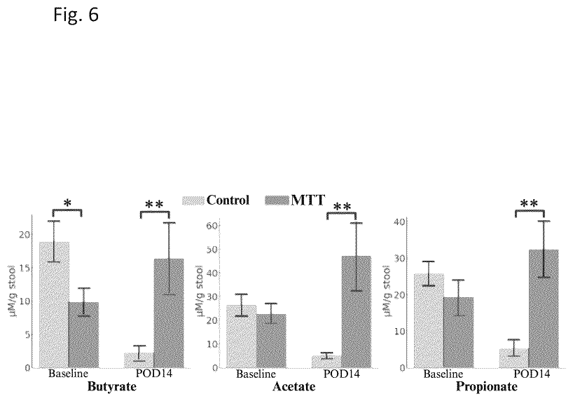

SCFAs (acetate, propionate, butyrate, and valerate) declined sharply after surgery in both groups. At baseline, acetate, propionate, and valerate concentrations were similar between cohorts, whereas butyrate levels were significantly lower in the MTT group (P=0.037). Despite these baseline differences, postoperative trajectories demonstrated rapid convergence between groups. Early overshoot of SCFA levels in MTT recipients supports accelerated functional recovery rather than baseline group bias. By POD14, concentrations of all measured SCFAs were significantly higher in MTT recipients compared with non-MTT controls (acetate P=0.0025, propionate P=0.0003, butyrate P=0.0018, valerate P=0.0069), indicating accelerated early recovery of microbial metabolic function (FIG. 6, Table 4).

TABLE 4
MTT restores SCFA production.

| Cohort | Time Point | Acetate | Propionate | Butyrate | Valerate |
|---|---|---|---|---|---|
| MTT | Baseline | 22.73 (14.03)^{a} | 19.28 (17.33)^{a} | 9.86 (6.86)^{b} | 2.72 (2.04)^{a} |
| | Inpatient^{†} | 8.21 (8.13)^{b} | 1.60 (2.47)^{b} | 0.44 (0.93)^{b} | 0.06 (0.16)^{b} |
| | POD14 | 47.16 (50.01)^{c} | 32.38 (26.35)^{c} | 16.36 (18.33)^{c} | 2.72 (3.70)^{a} |
| | Blanks | 0.15 (0.08) | 0.03 (<0.01) | 0.01 (<0.01) | 0.01 (<0.01) |
| Non-MTT | Baseline | 26.41 (17.94)^{a} | 25.68 (13.34)^{a} | 18.89 (12.06)^{a} | 3.70 (2.21)^{a} |
| Controls | POD14 | 5.06 (4.90)^{b} | 5.31 (8.41)^{b} | 2.21 (4.23)^{b} | 0.27 (0.54)^{b} |
| | Blanks | 1.25 (0.12) | 0.04 (0.01) | 0.01 (<0.01) | 0.01 (0.01) |

Fecal short-chain fatty acid (SCFA) concentrations (μM/g) in patients who received MTT versus non-MTT controls at baseline, while inpatient (transplant patients only), and POD14.
Values represent mean (standard deviation).
Superscript letters denote statistical comparisons within each SCFA; values sharing a letter are not significantly different (P > 0.05, ANOVA with post hoc test).
^{†}Between 42-96 hours postoperatively.

Branched-chain fatty acids (BCFAs; 2-methylbutyrate, isobutyrate, and isovalerate), which are derived from branched-chain amino acid fermentation, were significantly lower in the MTT group at baseline (P=0.008, P=0.018, and P=0.011, respectively; Table 5). By POD14, each BCFA was significantly higher in MTT recipients compared with non-MTT controls (2-methylbutyrate P=0.010, isobutyrate P=0.0049, isovalerate P=0.0086), demonstrating that MTT accelerates recovery of both carbohydrate- and protein-derived microbial fermentation products following surgery.

TABLE 5
Microbiota Transplant Therapy restores BCFA production.

| Cohort | Time Point | 2-Methylbutyrate | Isobutyrate | Isovalerate |
|---|---|---|---|---|
| Microbial | Baseline | 1.21 (0.85)^{a} | 2.27 (1.46)^{a} | 1.68 (1.18)^{a} |
| Transplant | Inpatient^{†} | 0.12 (0.12)^{b} | 0.41 (0.70)^{b} | 0.36 (0.62)^{b} |
| Therapy | POD14 | 1.28 (1.14)^{a} | 2.70 (1.72)^{a} | 1.74 (1.38)^{a} |
| | Blanks | 0.03 (0.01) | 0.03 (0.03) | <0.01 (<0.01) |
| Non-MTT | Baseline | 2.18 (0.79)^{c} | 5.20 (3.54)^{c} | 3.40 (1.76)^{c} |
| Controls | POD14 | 0.39 (0.69)^{b} | 0.78 (1.36)^{b} | 0.68 (1.17)^{b} |
| | Blanks | 0.01 (<0.01) | 0.02 (<0.01) | 0.01 (<0.01) |
| Microbial | Baseline | 1.21 (0.85)^{a} | 2.27 (1.46)^{a} | 1.68 (1.18)^{a} |
| Transplant | Inpatient^{†} | 0.12 (0.12)^{b} | 0.41 (0.70)^{b} | 0.36 (0.62)^{b} |
| Therapy | POD14 | 1.28 (1.14)^{a} | 2.70 (1.72)^{a} | 1.74 (1.38)^{a} |
| | Blanks | 0.03 (0.01) | 0.03 (0.03) | <0.01 (<0.01) |
| Non-MTT | Baseline | 2.18 (0.79)^{c} | 5.20 (3.54)^{c} | 3.40 (1.76)^{c} |
| Controls | POD14 | 0.39 (0.69)^{b} | 0.78 (1.36)^{b} | 0.68 (1.17)^{b} |
| | Blanks | 0.01 (<0.01) | 0.02 (<0.01) | 0.01 (<0.01) |

Fecal branched-chain fatty acid (BCFA) concentrations (μM/g) in patients who received MTT versus non-MTT controls at baseline, while inpatient (transplant patients only), and POD14.
Values represent mean (standard deviation).
Superscript letters denote statistical comparisons within each BCFA; values sharing a letter are not significantly different (P > 0.05, ANOVA with post hoc test).
^{†}Between 42-96 hours postoperatively. Of 420 measurements among all measured fatty acids in Tables 4 and 5, 8 (2%) were greater than 2 standard deviations from the mean and excluded from the calculations.

Antibiotic Exposure During 6-month Follow up Period

Given the potential impact of antibiotics on microbial engraftment, postoperative antibiotic exposure was prospectively recorded during the six-month follow-up period. Five of twelve patients in the MTT group received antibiotics after surgery.

One patient received a 48-hour course of piperacillin-tazobactam initiated on POD 9 for postoperative leukocytosis in the absence of radiographic evidence of intra-abdominal infection. A second patient received a single dose of amoxicillin-clavulanate on POD9 for wound drainage subsequently confirmed to represent a seroma. Two patients with stage III colon cancer received a single prophylactic dose of cefazolin at 10 and 12 weeks postoperatively during chemotherapy port placement. One additional patient was treated with a two-week course of amoxicillin-clavulanate for pancreatitis occurring seven weeks after surgery. No prolonged or repeated antibiotic exposure overlapped with primary sampling windows.

### Integrated Microbiome-Metabolite Recovery Signatures

To understand how the gut microbiome and its metabolic activity recover together during the perioperative period, we jointly analyzed bacterial composition and SCFA concentrations at three timepoints: before MTT (baseline), POD2, and POD14. To identify shared patterns linking microbial shifts with metabolic output, we applied partial least squares (PLS) modeling, which extracts latent axes capturing the dominant shared variation between two high-dimensional datasets.

Across all perioperative timepoints, a single dominant latent axis (Z2) consistently linked microbial community structure with SCFA output, representing the strongest shared microbe-metabolite covariance. This pattern was not evident at baseline, where microbial composition showed limited association with SCFA levels. By POD2, early associations between microbial shifts and SCFA production began to emerge, suggesting improving microbiome-metabolite coupling early after surgery. By POD14, Z2 early associations robustly predicted increases across both unbranched and branched SCFAs, suggesting strong microbiome-metabolite coupling during late recovery.

Z2 was positively loaded by fermentative genera, including Parvimonas, Peptostreptococcus, Butyricicoccus, and Intestinibacter, and inversely associated with Staphylococcus and Massiliimalia (Table 6). Together, these findings indicate that postoperative microbial reconstitution becomes progressively coupled to metabolic recovery across perioperative timepoints.

TABLE 6
Top genus drivers of Z2 by time window.

| | Positive Z2 Drivers | Negative Z2 Drivers |
|---|---|---|
| Window | (High Positive Loadings) | (High Negative Loadings) |
| Overall | Parvimonas, Peptostreptococcus, | Neglecta, Raoultibacter, Massiliimalia, |
| | Solobacterium, Intestinibacter, | Staphylococcus |
| | Butyricicoccus | |
| POD0 | Blautia, Bulleidia, Campylobacter, | Clostridium IV, Clostridium XIVa, |
| | Escherichia/Shigella, Lachnospira | Streptococcus, Treponema |
| POD2 | Lactiplantibacillus, Oscillibacter, | Allobaculum, Anaerostipes, Romboutsia, |
| | Peptostreptococcus, Enterocloster, | Succiniclasticum, Acidaminococcus |
| | Dielma | |
| POD14 | Veillonella, Intestinimonas, | Clostridiales, Dialister, Eisenbergiella, |
| | Lactobacillus, Massiliprevotella, | Sutterellaceae, Tyzzerella |
| | Streptococcus | |

Genera shown are the highest-magnitude (absolute) Z2 loading features from the 16S↔SCFA fusion PLS model within each time window. “Positive” and “Negative” indicate the sign of each genus's Z2 loading. Positive Z2 loadings reflect taxa that covary with the Z2 latent axis in the same direction as SCFA rate patterns captured in that window, whereas negative loadings reflect taxa that covary in the opposite direction.

MTT is Associated with Dynamic Remodeling of the Innate Immune Compartment at POD3

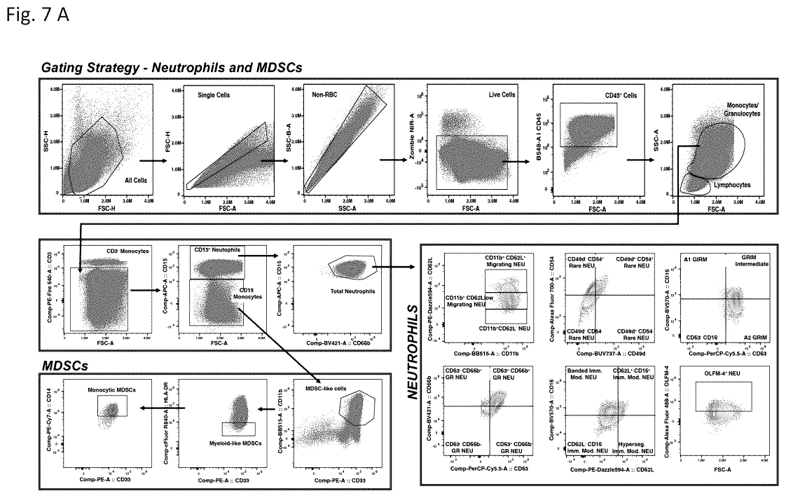

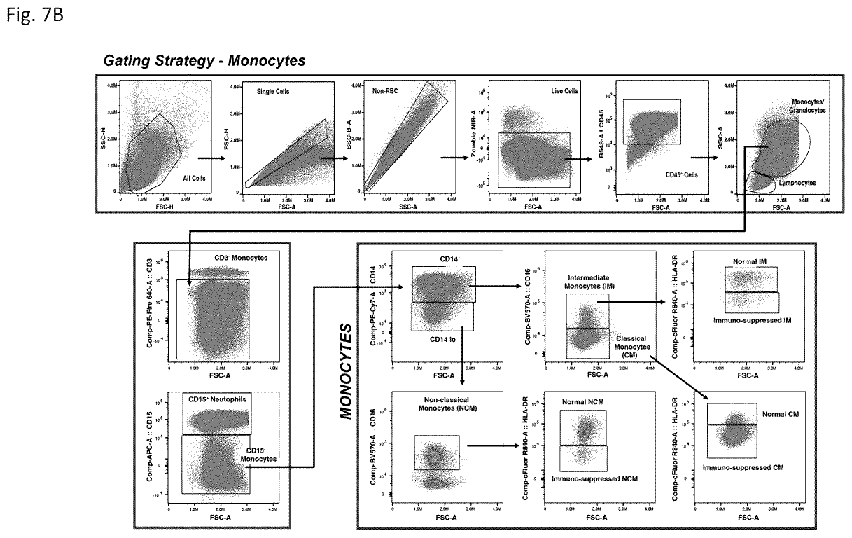

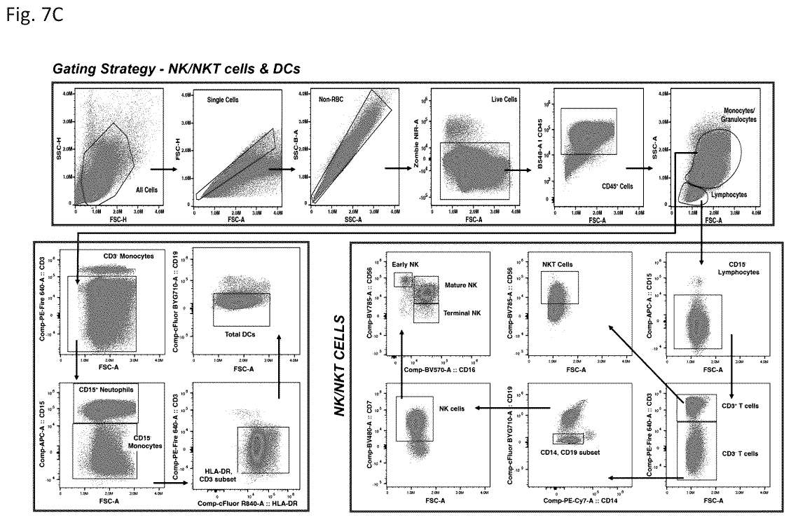

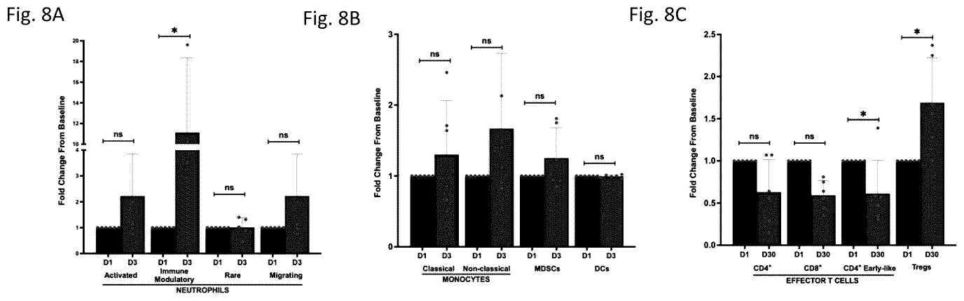

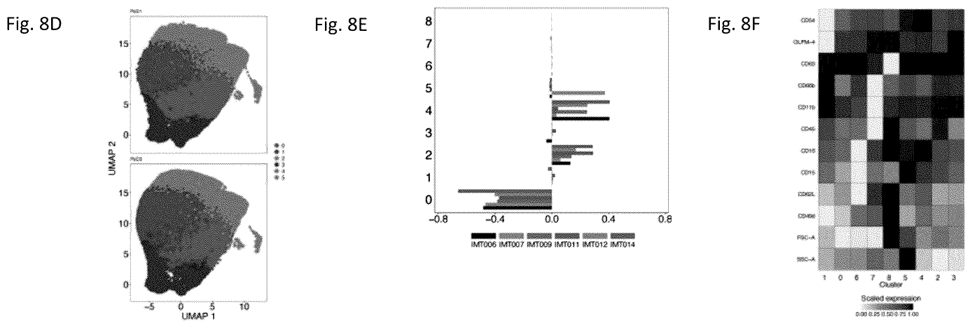

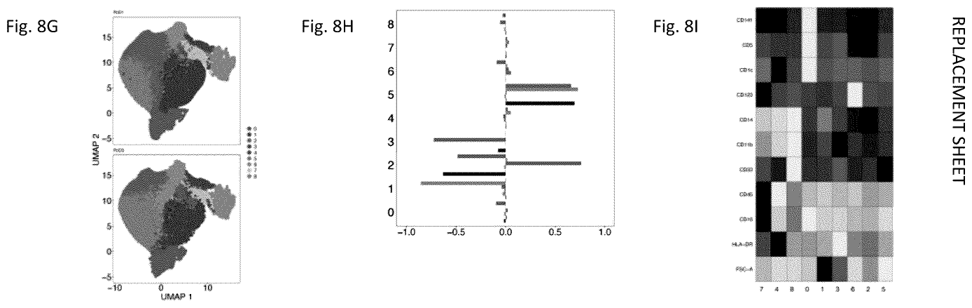

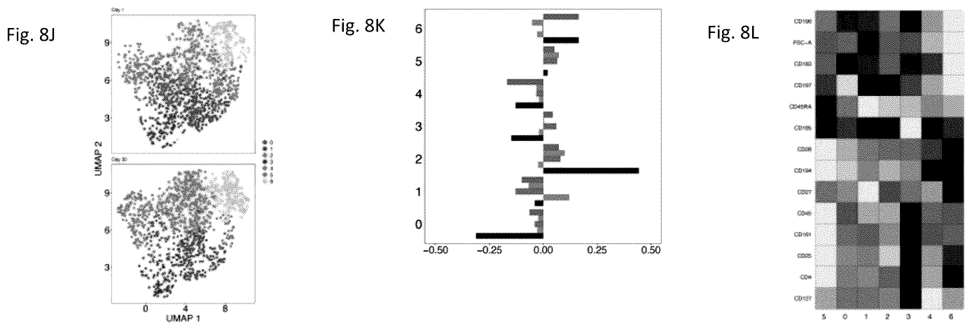

Having identified a progressively structured microbe-metabolite recovery trajectory, we next examined whether early postoperative immune dynamics reflected parallel remodeling within the innate compartment. MTT POD3 samples demonstrated marked remodeling of the innate immune compartments compared with POD1 MTT baseline samples (gating strategy shown in FIG. 7A). A 0.43-fold decrease was observed in CD62L⁻CD16⁻ immune-modulatory neutrophils. (35) In contrast, increased frequencies were detected in multiple neutrophil subsets, including CD63⁻CD66b⁺ granule-releasing neutrophils (1.53-fold), CD63⁻CD66b⁻ granule-releasing neutrophils (2.57-fold), CD11b⁺CD62L⁻ neutrophils (1.79-fold), CD11b⁺CD62L⁺ activated migrating neutrophils (2.23-fold) (FIG. 8A), and CD49d+CD54-rare neutrophils (6.24-fold). (35)

A pronounced expansion was observed among immature and immune-associated neutrophil populations, including banded neutrophils (10.06-fold), A1 GRIM neutrophils (11.13-fold), CD62L⁺CD16⁺ neutrophils (12.77-fold), and GRIM intermediate neutrophils (71.77-fold) (FIG. 8A). In parallel, POD3 samples demonstrated increased frequencies of myeloid-like myeloid-derived suppressor cells (MDSCs) (1.67-fold) and early natural killer (NK) cells (2.71-fold) compared with POD1 MTT baseline samples (FIG. 8B). No significant changes were observed in conventional monocyte or dendritic cell subsets at this time point (FIG. 8B; gating strategy shown in FIGS. 7B and 7C).

### MTT Alters Neutrophil Phenotypic Heterogeneity and Cluster Composition

Unsupervised clustering analysis of neutrophils identified nine distinct phenotypic clusters with unique marker expression profiles (FIG. 8D). These clusters encompassed populations characterized by quiescent phenotypes (CD62L\hat{ }high, CD11b\hat{ }low), (36) primed and activated effector states marked by degranulation (CD66b\hat{ }high), (37) activation (CD62L\hat{ }low), (38) and hyper-adhesive features (CD54\hat{ }high), (39) as well as clusters defined by banded, A1 GRIM, and CD62L⁺CD16⁺ phenotypes.

Dimensionality reduction using Uniform Manifold Approximation and Projection (UMAP) demonstrated distinct shifts in neutrophil composition at POD3 compared with POD1 MTT samples. Quantitative analysis of cluster proportions (FIG. 8E) and marker expression heatmaps (FIG. 8F) show a significant reduction in cluster c0, characterized by low expression of CD66b and CD62L. This reduction was accompanied by increased proportions of clusters c2 and c4, which were defined by CD62L+CD16⁺ marker expression. (38)

### Monocyte and Dendritic Cell Subset Remodeling Following MTT

Unsupervised clustering of monocytes and dendritic cells identified nine clusters with distinct phenotypic profiles (FIG. 8G). Comparative analysis of cluster proportions (FIG. 8H) and marker expression heatmaps (FIG. 8I) demonstrated a significant increase in the frequency of the C5 cluster, characterized by CD11b⁺CD14⁺HLA-DR\hat{ }low/− myeloid-derived suppressor cells, at POD3 relative to POD1 MTT baseline samples. This shift was accompanied by reduced frequencies of cross-presenting dendritic cell clusters (CD141⁺BDCA3⁺), (40) including clusters C1, C2, and C3.

### MTT Promotes Adaptive Immune Remodeling at POD30

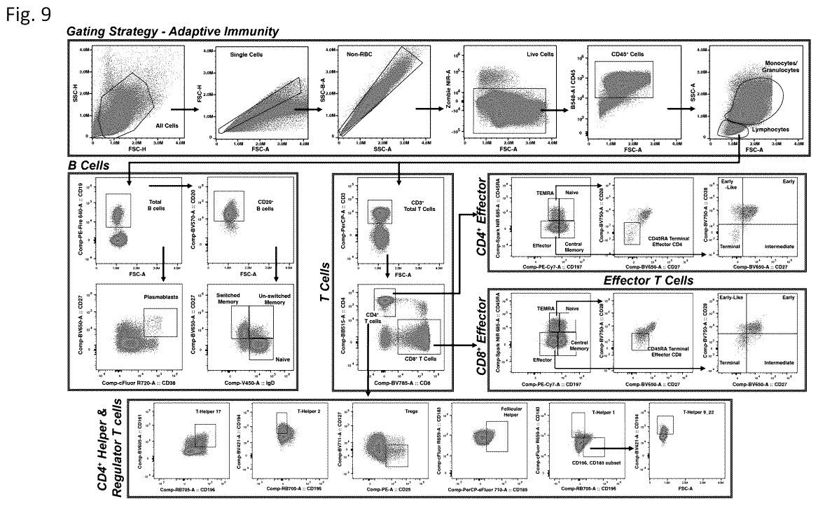

Having established early innate immune reprogramming following MTT, we next examined whether these changes were followed by coordinated remodeling of the adaptive immune compartment during later postoperative recovery (gating strategy shown in FIG. 9). Immune profiling of POD30 MTT samples revealed a reduction in terminally differentiated and effector T cell populations compared with POD1 MTT baseline samples. These included CD8⁺ terminal effector T cells (0.52-fold), total CD8⁺ effector T cells (0.61-fold), CD4⁺ effector subsets (0.63-fold), and Th1 cells (0.69-fold) (FIG. 8C). In contrast, naive CD4⁺ T cells increased by 2.36-fold, while early and intermediate CD4⁺ effector subsets increased by 2.73-fold and 16.57-fold, respectively. Regulatory T cells (Tregs) increased by 1.69-fold (FIG. 8C).

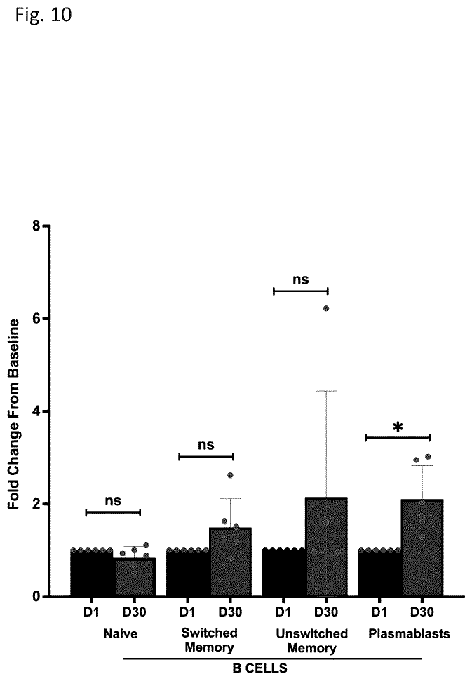

Among B cell populations, plasmablast frequencies increased by 2.11-fold (FIG. 10). Substantial increases were additionally observed in γδ T cell subsets, including CD197⁺CD45RA⁻ (3.07-fold) and CD197⁺CD45RA⁺ (4.01-fold) populations, (41) as well as in unswitched memory B cells (3.70-fold) in POD30 MTT samples relative to POD1 MTT baseline samples.

### Unsupervised Clustering Reveals Adaptive Immune Reorganization Following MTT

Unsupervised clustering analysis of Tregs identified seven phenotypically and functionally distinct clusters (FIG. 8J). Quantitative analysis demonstrated an expansion of the C2 and C4 Treg clusters that suggest migratory potential indicated by higher expression of CXCR4 (CD185, C2) and CCR4 (CD194, C6) at POD30, resembling follicular Tregs implicated in regulation of immune responses to self-antigens. This expansion was accompanied by a reduction in the Naïve/Resting-like Treg⁺ C0 cluster, characterized by reduced expression of CD28 and CD196 (FIG. 8K and FIG. 8L).

### Cross-Omic Integration Identifies Time-Stratified Associations Following MTT

After identifying a consistent gut recovery axis (Z2) linking microbial composition and SCFA production across perioperative timepoints, and observing staged immune remodeling, we next asked whether these immune dynamics tracked with the same gut recovery trajectory at the level of individual patients. To examine this, we integrated microbiome composition, SCFA concentrations, and immune-cluster frequency changes using a Shared-Embedding Correlation Network (SECN) framework. This approach derives low-rank patient embeddings that capture shared cross-domain variation and constructs sparse correlation networks to stabilize inference in small-n longitudinal datasets. Rather than evaluating each dataset independently, this framework enables identification of structured cross-omic associations across systems and time.

When relationships were examined across perioperative windows, a time-stratified pattern emerged. Early after surgery, innate-associated immune features demonstrated stronger statistical coupling with gut microbial and metabolite shifts. At later timepoints, adaptive-associated immune features exhibited relatively stronger cross-domain associations. Time-stratified shared-embedding correlation networks integrating genus-level microbiome composition, SCFA dynamics, and immune cluster deltas across perioperative recovery were determined. Early postoperative networks were enriched for associations between gut-derived features and innate immune remodeling, whereas later networks showed increased coupling with adaptive immune features, consistent with the temporal immune structure. Induced connector backbones highlighted three-view connector nodes that bridge microbial, metabolic, and immune layers. The induced subgraph illustrated compact cross-omic motifs linking gut recovery features with systemic immune remodeling. This temporal pattern paralleled the independently observed immune remodeling sequence, in which innate transitions preceded adaptive changes. Together, these findings indicate that microbial and metabolic recovery trajectories are temporally aligned with staged systemic immune remodeling, with distinct immune compartments predominating at different phases of postoperative recovery.

### Lead-lag Testing Identifies Time-Ordered Innate to Adaptive Associations

To test whether early innate immune changes preceded later adaptive remodeling, we performed lead-lag analysis linking innate deltas at one timepoint to subsequent adaptive deltas while adjusting for baseline adaptive levels. This approach allowed us to quantify directional associations between immune compartments across perioperative windows.

Across both POD0→POD1 and POD1→POD14 windows, we identified structured innate→adaptive association networks (Table 7). Among adaptive targets, CD4 T cells (n=388), CD8 T cells (n=326), and B cells (n=238) exhibited the highest in-degree connectivity. Among innate sources, monocytes (n=348), neutrophils (n=348), and NK/NKT populations (n=256) showed the strongest out-degree contributions, consistent with innate-linked changes preceding adaptive transitions.

TABLE 7
Lead-lag innate-to-adaptive immune edge counts by time window.

| | Positive | Negative | Total |
|---|---|---|---|
| Lead-lag window | correlations (n) | correlations (n) | edges (n) |
| POD0→POD1 | 267 | 209 | 476 |
| POD1→POD14 | 267 | 209 | 476 |

For each lead-lag window (POD0→POD1 and POD1→POD14), the table reports the number of significant innate → adaptive edges that pass effect-size (|ρ| ≥ 0.30), BH-FDR control (q ≤ 0.10), and sparsification criteria. Values are derived from the baseline-adjusted (partial Spearman) analysis, in which innate deltas at the lead timepoint predict adaptive deltas at the subsequent timepoint after adjusting for adaptive baseline at the lead timepoint.

Although total edge counts were similar across windows due to fixed sparsification criteria, the identity and sign of retained edges differed across phases, indicating window-specific association structure. We next examined whether the strength of cross-omic coupling varied by perioperative phase. Using paired comparisons of absolute correlation magnitudes across feature pairs (n=350), median |ρ| increased from 0.354 at D0 to 0.393 at D1, before decreasing to 0.314 at D14. |ρ| was significantly higher at D1 versus D0 (paired Wilcoxon P=0.035) and D1 versus D14 (P=0.0013), indicating a transient early increase in cross-domain association strength.

### Connector-node Analysis Identifies Multimodal Bridges Across Microbial, SCFA, and Immune Layers

To characterize features that bridge modalities, we identified three-view connector nodes, defined as nodes with neighbors in all three layers (immune, microbe, and SCFA). Connector backbones were examined using induced subgraphs to highlight compact cross-omic motifs linking immune remodeling to gut recovery features. Induced connector backbones highlighted three-view connector nodes that bridge microbial, metabolic, and immune layers. The induced subgraph illustrated compact cross-omic motifs linking gut recovery features with systemic immune remodeling. The composition of connector-node neighborhoods was determined, and showed heterogeneous connectivity across immune, microbial, and SCFA layers. Connector nodes represented features with statistically supported associations spanning microbial, SCFA, and immune modalities. Notably, three-view connectivity was concentrated among non-immune features (Fisher's exact test OR=0.28, 95% CI 0.12-0.63, P=7.97×10⁻⁴; Table 8), highlighting microbial and SCFA components as the dominant cross-layer “bridge” elements under the current sparsification and significance thresholds, with a subset of immune features participating in these multimodal links.

TABLE 8
Immune versus non-immune feature representation
among three-view connector nodes.

| | Non-connector nodes | Connector nodes | Row total |
|---|---|---|---|
| Non-immune features | 59 | 30 | 89 |
| Immune features | 33 | 17 | 50 |

Counts are shown for immune and non-immune features that are connectors (nodes with neighbors in all three views: immune, SCFA, microbe) versus non-connectors. Fisher's exact test indicates immune features are depleted among connectors (OR = 0.28, 95% CI 0.12-0.63, P = 7.97 × 10⁻⁴).

| | Non-connector nodes | Connector nodes | Row total |
|---|---|---|---|
| Non-immune features | 44 | 38 | 82 |
| Immune features | 50 | 12 | 62 |
| Column total | 94 | 50 | 144 |

Non-immune features are preferentially enriched among three-view connector nodes, indicating that cross-omic “bridge” roles in this network are primarily carried by microbial and SCFA features (with a subset of immune features contributing) under current thresholds.

### Discussion

In this Phase I clinical study, we show that MTT administered during the early postoperative period is associated with a coordinated, time-ordered recovery program spanning microbial community structure, microbial metabolic function, and systemic immune remodeling following colorectal surgery. Direct interrogation of integrated microbial, metabolic, and immune recovery trajectories in surgical patients has been limited. (2, 16, 17) MTT promoted durable donor engraftment, accelerated restoration of short-chain and branched-chain fatty acids, and was accompanied by distinct innate and adaptive immune transitions that unfolded sequentially across the postoperative course. In this setting, MTT functions not only as a potential therapeutic strategy but also as a controlled biological perturbation that enables interrogation of recovery biology in human surgical patients. (34)

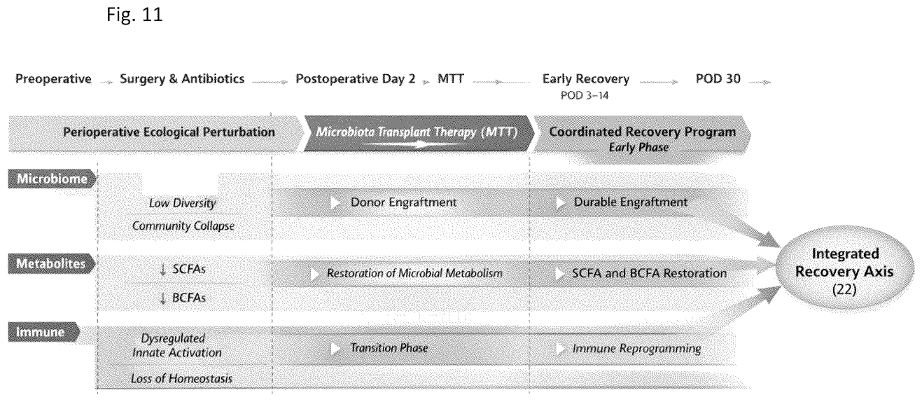

A central observation of this study is the dissociation between early recovery of microbial metabolic function and the slower restoration of canonical SCFA-producing taxa. Early after MTT, enrichment of Akkermansia and Collinsella was observed despite incomplete recovery of classical butyrate-producing genera such as Blautia, Ruminococcaceae, and Lachnospiraceae. Notably, accelerated SCFA recovery occurred despite lower baseline levels in MTT recipients, further supporting a treatment-associated effect driven by network reconstitution rather baseline than compositional differences. Although Akkermansia and Collinsella are not primary SCFA producers, both taxa occupy critical upstream positions in the gut metabolic network. (42-44) Akkermansia resides within the mucus layer and degrades mucin into oligosaccharides and organic acids that serve as substrates for downstream fermentative taxa, (42, 43) while Collinsella ferments host- and diet-derived carbohydrates into lactate, formate, and other intermediates that feed pathways supporting butyrate and propionate production. (44, 45) The early expansion of these taxa after MTT likely conditions the gut environment and provides essential feedstock for SCFA synthesis, thereby enabling accelerated metabolic recovery even before full reconstitution of downstream producers. (46) These coordinated dynamics are summarized schematically in FIG. 11.

This network-based interpretation is further supported by the observation that SCFA recovery occurred despite only modest, non-significant differences in the abundance of canonical butyrate-producing taxa between MTT recipients and non-MTT controls at POD14 and POD30. SCFA concentrations reflect emergent properties of microbial ecosystems, integrating substrate availability, cross-feeding efficiency, and metabolic activity rather than simple taxonomic presence. (47) Early enrichment of upstream suppliers may therefore increase SCFA output by enhancing the activity of downstream producers, accelerating their functional recovery trajectory, and reshaping metabolic interactions across the community. These findings emphasize that functional readouts capture recovery dynamics that are not apparent from abundance-based analyses alone. (47, 48)

Consistent with this interpretation, integrated microbiome-metabolite modeling identified a dominant latent recovery axis (Z2) that coherently linked microbial community structure to restoration of both unbranched and branched SCFA. Z2 exhibited weak and inconsistent coupling to SCFAs at baseline, began to align with fermentative metabolites by POD2, and by POD14 robustly predicted increases across all measured SCFA. Taxa with strong positive loadings on Z2 included fermentative genera associated with SCFA production, whereas taxa inversely associated with Z2 included organisms less aligned with fermentative metabolism. In contrast, a separate latent axis reflecting baseline community structure showed minimal association with metabolic recovery. Reducing high-dimensional microbial features to interpretable latent axes provides a tractable framework for modeling postoperative recovery as a system-level process.

Beyond the gut ecosystem, MTT was associated with dynamic remodeling of the systemic immune compartment that followed a temporally structured pattern. Early after transplantation, POD3 samples demonstrated marked increased heterogeneity consistent with coordinated innate immune remodeling across neutrophil subsets. Expansion of granule-releasing and CD11b⁺ migratory neutrophils is consistent with enhanced degranulation and tissue trafficking, while enrichment of banded, GRIM intermediate, and A1 GRIM neutrophils reflects emergency granulopoiesis and accelerated neutrophil maturation under systemic inflammatory stress. (18, 49-54) Importantly, these inflammatory features were accompanied by parallel expansion of immunomodulatory neutrophil subsets and myeloid-derived suppressor cells, suggesting that innate immune activation was counterbalanced by emerging regulatory programs rather than progressing toward unchecked inflammation. (52, 55, 56) The concomitant increase in early natural killer cells further supports coordinated activation across innate immune compartments. (57) Together, this immune profile reflects a tightly regulated response that balances antimicrobial defense, tissue protection, and compensatory immune restraint during early postoperative recovery. Notably, these innate immune transitions unfolded in temporal alignment with microbial engraftment and recovery of fermentative metabolism, supporting a model in which immune remodeling tracks restoration of gut microbial function following surgery.

At later timepoints, immune remodeling shifted toward features consistent with resolution and immune homeostasis. By POD30, MTT recipients exhibited contraction of terminally differentiated effector and Th1 T-cell populations, accompanied by expansion of naïve and early or intermediate CD4⁺ effector subsets, indicating active immune reconstitution with preserved differentiation plasticity. (58, 59) Enrichment of γδ T-cell populations supports enhanced innate-like immune surveillance and regulatory bridging between innate and adaptive immunity. (60-62) In parallel, increased plasmablasts and unswitched memory B cells point to active humoral engagement and B-cell-mediated tissue repair processes. (63-65) Collectively, these adaptive immune changes align temporally with microbial engraftment and metabolic recovery and are consistent with immune resolution rather than immune exhaustion.

An important limitation in interpreting treatment-associated multi-omic differences is the open-label design and the use of matched non-MTT controls drawn from a prior study. As a result, sequencing, metabolomics, and immune assays may not be fully contemporaneous across groups and could reflect run- and calendar-time differences in addition to biological effects. Such non-overlap can, in principle, influence apparent Bray-Curtis separation (FIG. 1B), shift absolute SCFA/BCFA concentrations (FIG. 6; Tables 4-5), and affect edge retention and connector structure in integrated networks. To mitigate these risks, multi-omics integration was performed using conservative stability-selection and sparsification strategies designed to reduce false discovery under small-n constraints, and immune analyses were anchored in prespecified within-subject contrasts (POD1→POD3 for innate; POD1→POD30 for adaptive) to emphasize remodeling over baseline abundance.

A second limitation is that immune phenotyping was performed in MTT recipients only, while controls contributed microbiome and metabolite data. Accordingly, the innate and adaptive immune trajectories described here reflect remodeling within the treated cohort and cannot be directly compared against standard postoperative recovery in non-transplanted patients. Validation in contemporaneous non-MTT immune controls will be helpful in determining which immune features are intervention-associated versus common postoperative kinetics. In addition, sampling windows were not fully synchronized across stool and blood modalities (e.g., microbiome/SCFA emphasis at baseline, POD2, POD14 versus immune contrasts at POD3 and POD30). This partial misalignment limits inference about exact temporal precedence across systems; therefore, we interpret the integrated findings as time-ordered coordination with early innate remodeling aligning with the emergence of microbe-metabolite coupling, followed by later adaptive transitions consistent with resolution, rather than as a precise causal timeline. Furthermore, microbiome and metabolomic sampling began at POD14 for the primary postoperative window, limiting our ability to directly assess whether very early microbial dynamics contribute to the early clinical recovery signals observed in this cohort. Denser early postoperative sampling will be required in future trials to determine whether microbial shifts precede or accompany early improvements in clinical recovery metrics. Finally, our integrated modeling approach was selected for interpretability and stability under small-n longitudinal constraints. Broader benchmarking against alternative multi-omic frameworks (e.g., MOFA+, DIABLO, dynamic Bayesian networks, Granger-style approaches, or related temporal graph methods) was beyond the scope of the current Phase I dataset and will be more appropriately addressed in larger cohorts where model complexity can be evaluated against independent validation targets.

In summary, this study supports a coordinated, time-ordered recovery program across microbial, metabolic, and immune systems in MTT recipients following colorectal surgery (FIG. 11). By applying a microbiome-targeted intervention as a biological probe in surgical patients, this work extends postoperative recovery research beyond descriptive associations and into mechanistically interpretable human systems.

### DOCUMENTS CITED IN EXAMPLE 1

1. E. K. Abernethy, E. H. Aly, Postoperative Ileus after Minimally Invasive Colorectal Surgery: A Summary of Current Strategies for Prevention and Management. Dig Surg 41, 79-91 (2024).
2. J. Barbieux et al., Does enhanced recovery reduce postoperative ileus after colorectal surgery? J Visc Surg 154, 79-85 (2017).
3. S. J. Chapman, A. Pericleous, C. Downey, D. G. Jayne, Postoperative ileus following major colorectal surgery. Br J Surg 105, 797-810 (2018).
4. Gaudilliere B et al., Clinical recovery from surgery correlates with single-cell immune signatures. Sci Transl Med 5, 255ra131 (2014).
5. E. Mazzotta, E. C. Villalobos-Hernandez, J. Fiorda-Diaz, A. Harzman, F. L. Christofi, Postoperative Ileus and Postoperative Gastrointestinal Tract Dysfunction: Pathogenic Mechanisms and Novel Treatment Strategies Beyond Colorectal Enhanced Recovery After Surgery Protocols. Front Pharmacol 11, 583422 (2020).
6. B. D. Shogan et al., Collagen degradation and MMP9 activation by Enterococcus faecalis contribute to intestinal anastomotic leak. Science Translational Medicine 7, 286ra268-286ra268 (2015).
7. C. A. Weiss et al., Six years of surgical wound infection surveillance at a tertiary care center: review of the microbiologic and epidemiological aspects of 20,007 wounds. Arch Surg 134, 1041-1048 (1999).
8. J. A. Jacobs, J. H. Tjhie, M. G. Smeets, C. S. Schot, L. M. Schouls, Genotyping by amplified fragment length polymorphism analysis reveals persistence and recurrence of infection with Streptococcus anginosus group organisms. J Clin Microbiol 41, 2862-2866 (2003).
9. A. Alam et al., The microenvironment of injured murine gut elicits a local pro-restitutive microbiota. Nature Microbiology 1, (2016).
10. A. Alam, A. Neish, Role of gut microbiota in intestinal wound healing and barrier function. Tissue Barriers 6, 1539595 (2018).
11. R. J. Mulders et al., Microbiota in obesity: interactions with enteroendocrine, immune and central nervous systems. Obesity Reviews 19, 435-451 (2018).
12. J.-p. Furet et al., Effect of different bariatric operations on food tolerance and quality of eating. Obesity Surgery 20, 1-6 (2010).
13. B. D. Shogan et al., Alterations of the Rectal Microbiome Are Associated with the Development of Postoperative Ileus in Patients Undergoing Colorectal Surgery. Journal of Gastrointestinal Surgery 24, 1663-1672 (2020).
14. R. L. Nichols, P. Broido, R. E. Condon, S. L. Gorbach, L. M. Nyhus, Effect of preoperative neomycin erythromycin intestinal preparation on the incidence of infectious complications following colon surgery. Annals of Surgery 178, 453-462 (1973).
15. L. Koskenvuo et al., Morbidity After Mechanical Bowel Preparation and Oral Antibiotics Prior to Rectal Resection: The MOBILE2 Randomized Clinical Trial. JAMA Surg, (2024).
16. J. Kohn et al., The Role of Surgical and Perioperative Factors in Shaping Gut Microbiome Recovery After Colorectal Surgery. Antibiotics (Basel) 14, 881 (2025).
17. H. Nalluri-Butz et al., A pilot study demonstrating the impact of surgical bowel preparation on intestinal microbiota composition following colon and rectal surgery. Sci Rep 12, 10559 (2022).
18. A. N. Connelly et al., Optimization of methods for the accurate characterization of whole blood neutrophils. Sci Rep 12, 3667 (2022).
19. S. Boatman et al., Diet-induced shifts in the gut microbiota influence anastomotic healing in a murine model of colonic surgery. Gut Microbes 15, 2283147 (2023).
20. S. Boatman et al., Anastomotic leak occurs independently from microbiota shifts associated with surgical bowel preparation. Sci Rep 14, 21711 (2024).
21. S. Gaines et al., Western Diet Promotes Intestinal Colonization by Collagenolytic Microbes and Promotes Tumor Formation After Colorectal Surgery. Gastroenterology 158, 958-970.e952 (2020).
22. B. D. Shogan et al., Intestinal anastomotic injury alters spatially defined microbiome composition and function. Microbiome 2, 1-10 (2014).
23. K. Atarashi et al., Ectopic colonization of oral bacteria in the intestine drives T (H) 1 cell induction and inflammation. Science 358, 359-365 (2017).
24. Z. M. Burcham et al., Patterns of Oral Microbiota Diversity in Adults and Children: A Crowdsourced Population Study. Sci Rep 10, 2133 (2020).
25. B. J. Kunath, C. De Rudder, C. C. Laczny, E. Letellier, P. Wilmes, The oral-gut microbiome axis in health and disease. Nat Rev Microbiol, (2024).
26. D. Moutsoglou et al., Novel Microbial Engraftment Trajectories following Microbiota Transplantation Therapy in Ulcerative Colitis. J Crohns Colitis, (2024).
27. C. Staley et al., Lower endoscopic delivery of freeze-dried intestinal microbiota results in more rapid and efficient engraftment than oral administration. Scientific Reports 11, 1-9 (2021).
28. C. Staley et al., Successful Resolution of Recurrent Clostridium difficile Infection using Freeze-Dried, Encapsulated Fecal Microbiota; Pragmatic Cohort Study. American Journal of Gastroenterology 112, 940-947 (2017).
29. C. Staley et al., Predicting recurrence of Clostridium difficile infection following encapsulated fecal microbiota transplantation. Microbiome 6, 166-166 (2018).
30. C. Staley et al., Durable long-term bacterial engraftment following encapsulated fecal microbiota transplantation to treat <i>Clostridium difficile</i> infection. mBio 10, e01586-01519 (2019).
31. C. Staley et al., Encapsulated Fecal Microbiota Transplantation To Treat Clostridium difficile Infection. mBio 23, e01586-01519 (2019).
32. C. Staley et al., Community dynamics drive punctuated engraftment of the fecal microbiome following transplantation using freeze-dried, encapsulated fecal microbiota. Gut Microbes 8, 276-288 (2017).
33. B. P. Vaughn et al., A pilot study of fecal bile acid and microbiota profiles in inflammatory bowel disease and primary sclerosing cholangitis. Clin Exp Gastroenterol 12, 9-19 (2019).
34. A. Khoruts, Targeting the microbiome: from probiotics to fecal microbiota transplantation. Genome Med 10, 80 (2018).
35. C. J. Schofield, R. Tirouvanziam, L. W. Garratt, OMIP-100: A flow cytometry panel to investigate human neutrophil subsets. Cytometry A 105, 81-87 (2024).
36. R. Spijkerman et al., Flow cytometric evaluation of the neutrophil compartment in COVID-19 at hospital presentation: A normal response to an abnormal situation. J Leukoc Biol 109, 99-114 (2021).
37. G. Ramakrishnan et al., Systemic neutrophil degranulation and emergency granulopoiesis in patients with Clostridioides difficile infection. Anaerobe 87, 102840 (2024).
38. S. H. Bongers et al., Kinetics of Neutrophil Subsets in Acute, Subacute, and Chronic Inflammation. Front Immunol 12, 674079 (2021).
39. S. Bissenova, D. Ellis, C. Mathieu, C. Gysemans, Neutrophils in autoimmunity: when the hero becomes the villain. Clin Exp Immunol 210, 128-140 (2022).
40. S. L. Jongbloed et al., Human CD141⁺ (BDCA-3)+ dendritic cells (DCs) represent a unique myeloid DC subset that cross-presents necrotic cell antigens. J Exp Med 207, 1247-1260 (2010).
41. M. Lawand, J. Dechanet-Merville, M. C. Dieu-Nosjean, Key Features of Gamma-Delta T-Cell Subsets in Human Diseases and Their Immunotherapeutic Implications. Front Immunol 8, 761 (2017).
42. P. Paone, P. D. Cani, Mucus barrier, mucins and gut microbiota: the expected slimy partners? Gut 69, 2232-2243 (2020).
43. S. Y. Geerlings, I. Kostopoulos, W. M. de Vos, C. Belzer, Akkermansia muciniphila in the Human Gastrointestinal Tract: When, Where, and How? Microorganisms 6, (2018).
44. L. F. Gomez-Arango et al., Low dietary fiber intake increases Collinsella abundance in the gut microbiota of overweight and obese pregnant women. Gut Microbes 9, 189-201 (2018).
45. A. Kageyama, Y. Benno, T. Nakase, Phylogenic and phenotypic evidence for the transfer of Eubacterium fossor to the genus Atopobium as Atopobium fossor comb. nov. Microbiol Immunol 43, 389-395 (1999).
46. L. Payling et al., The effects of carbohydrate structure on the composition and functionality of the human gut microbiota. Trends in food science & technology 97, 233-248 (2020-03).
47. E. J. Culp, A. L. Goodman, Cross-feeding in the gut microbiome: Ecology and mechanisms. Cell Host Microbe 31, 485-499 (2023).
48. M. A. Henson, P. Phalak, Suboptimal community growth mediated through metabolite crossfeeding promotes species diversity in the gut microbiota. PLOS Comput Biol 14, e1006558 (2018).
49. L. A. Cagle et al., Early mechanisms of neutrophil activation and transmigration in acute lung injury. Front Physiol 13, 1059686 (2022).
50. C. Rosales, Neutrophil: A Cell with Many Roles in Inflammation or Several Cell Types? Front Physiol 9, 113 (2018).
51. A. Hidalgo, E. R. Chilvers, C. Summers, L. Koenderman, The Neutrophil Life Cycle. Trends Immunol 40, 584-597 (2019).
52. F. Veglia, E. Sanseviero, D. I. Gabrilovich, Myeloid-derived suppressor cells in the era of increasing myeloid cell diversity. Nat Rev Immunol 21, 485-498 (2021).
53. B. Malengier-Devlies, M. Metzemaekers, C. Wouters, P. Proost, P. Matthys, Neutrophil Homeostasis and Emergency Granulopoiesis: The Example of Systemic Juvenile Idiopathic Arthritis. Front Immunol 12, 766620 (2021).
54. J. V. Camp, C. B. Jonsson, A Role for Neutrophils in Viral Respiratory Disease. Front Immunol 8, 550 (2017).
M. Peiseler, P. Kubes, More friend than foe: the emerging role of neutrophils in tissue repair. J Clin Invest 129, 2629-2639 (2019).
56. E. Kolaczkowska, P. Kubes, Neutrophil recruitment and function in health and inflammation. Nat Rev Immunol 13, 159-175 (2013).
57. U. C. Kucuksezer et al., The Role of Natural Killer Cells in Autoimmune Diseases. Front Immunol 12, 622306 (2021).
58. H. Chi, M. Pepper, P. G. Thomas, Principles and therapeutic applications of adaptive immunity. Cell 187, 2052-2078 (2024).
59. N. Caccamo, S. A. Joosten, T. H. M. Ottenhoff, F. Dieli, Atypical Human Effector/Memory CD4 (+) T Cells With a Naive-Like Phenotype. Front Immunol 9, 2832 (2018).
60. A. B. C. Cherry, N. A. Gherardin, H. I. Sikder, Intracellular radar: Understanding gammadelta T cell immune surveillance and implications for clinical strategies in oncology. Front Oncol 12, 1011081 (2022).
61. B. A. Tsz Kin Suen, Alice Scarpa, Anca Dorhoi, Mihai G Netea, Katarzyna Placek, A dual Nature of γδ T cell immune memory responses. Elife 14, (2025).
62. D. Z. Yun Shen, Liyou Qiu, Xioamin Lai, Meredith Simon, Ling Shen, Zhongchen Kou, Qifan Wang, Liming Jiang, Jim Estep, Robert Hunt, Michelle Clagett, Prabhat K Sehgal, Yunyaun Li, Xuejun Zeng, Craig T Morita, Michael B Brenner, Norman L Letvin, Zheng W Chen, Adaptive immune response of Vgamma2Vdelta2+ T cells during mycobacterial infections. Science 295, 2255-2258 (2002).
63. E. Ledoult et al., Dual role of plasmablasts as immune regulators or amplifiers in COVID-19. Clin Immunol 281, 110609 (2025).
64. R. F. Sirbulescu et al., Mature B cells accelerate wound healing after acute and chronic diabetic skin lesions. Wound Repair Regen 25, 774-791 (2017).
65. R. F. Sirbulescu et al., B cells support the repair of injured tissues by adopting MyD88-dependent regulatory functions and phenotype. FASEB J 35, e22019 (2021).
66. M. J. Hamilton, A. R. Weingarden, M. J. Sadowsky, A. Khoruts, Standardized frozen preparation for transplantation of fecal microbiota for recurrent Clostridium difficile infection. American Journal of Gastroenterology 107, 761-767 (2012).
67. L. Baietto et al., A 30-years Review on Pharmacokinetics of Antibiotics: Is the Right Time fo Pharmacogenetics? Current Drug Metabolism 15, 581-598 (2014).
68. J. G. Caporaso et al., Ultra-high-throughput microbial community analysis on the Illumina HiSeq and MiSeq platforms. ISME Journal 6, 1621-1624 (2012).
69. D. M. Gohl et al., Systematic improvement of amplicon marker gene methods for increased accuracy in microbiome studies. Nature Biotechnology 34, 942-949 (2016).
70. R. García-Villalba et al., Alternative method for gas chromatography-mass spectrometry analysis of short-chain fatty acids in faecal samples. Journal of Separation Science 35, 1906-1913 (2012).
71. S. Zhang, H. Wang, M. J. Zhu, A sensitive GC/MS detection method for analyzing microbial metabolites short chain fatty acids in fecal and serum samples. Talanta 196, 249-254 (2019).
72. P. D. Schloss et al., Introducing mothur: open-source, platform-independent, community-supported software for describing and comparing microbial communities. Applied and Environmental Microbiology 75, 7537-7541 (2009).
73. C. Staley et al., Predicting recurrence of Clostridium difficile infection following encapsulated fecal microbiota transplantation. Microbiome 6, 166-166 (2018).
74. E. Pruesse et al., SILVA: a comprehensive online resource for quality checked and aligned ribosomal RNA sequence data compatible with ARB. Nucleic Acids Research 35, 7188-7196 (2007).
75. S. M. Huse, D. M. Welch, H. G. Morrison, M. L. Sogin, Ironing out the wrinkles in the rare biosphere through improved OTU clustering. Environmental microbiology 12, 1889-1898 (2010).
76. R. C. Edgar, B. J. Haas, J. C. Clemente, C. Quince, R. Knight, UCHIME improves sensitivity and speed of chimera detection. Bioinformatics 27, 2194-2200 (2011).
77. S. L. Westcott, P. D. Schloss, OptiClust, an Improved Method for Assigning Amplicon-Based Sequence Data to Operational Taxonomic Units. mSphere 2, (2017).
78. J. R. Cole et al., The Ribosomal Database Project: improved alignments and new tools for RNA analysis. Nucleic Acids Research 37, D141-D145 (2009).
79. C. E. Shannon, W. Weaver, The Mathematical Theory of Communication. (The University of Illinois Press, Urbana, 1949), pp. 117-117.
80. J. R. Bray, J. T. Curtis, An Ordination of the Upland Forest Communities of Southern Wisconsin. Ecological Monographs 27, 325-325 (1957).
81. M. J. Anderson, T. J. Willis, Canonical analysis of principal coordinates: A useful method of constrained ordination for ecology. Ecology 84, 511-525 (2003).
82. K. R. Clarke, Non-parametric multivariate analyses of changes in community structure. Australian Journal of Ecology 18, 117-143 (1993).
83. D. Knights et al., Bayesian community-wide culture-independent microbial source tracking. Nature methods 8, 761-763 (2011).
84. N. A. Nguyen et al., A comprehensive assessment of four whole blood stabilizers for flow-cytometric analysis of leukocyte populations. Cytometry A 103, 313-324 (2023).
85. M. Spasic et al., Spectral Flow Cytometry Methods and Pipelines for Comprehensive Immunoprofiling of Human Peripheral Blood and Bone Marrow. Cancer Res Commun 4, 895-910 (2024).
86. J. Aitchison, The statistical analysis of compositional data. C. a. Hall, Ed., (1986).
87. G. B. Gloor, J. M. Macklaim, V. Pawlowsky-Glahn, J. J. Egozcue, Microbiome Datasets Are Compositional: And This Is Not Optional. Front Microbiol 8, 2224 (2017).
88. S. Wold, M. Sjostrom, L. Ericksson, PLS-regression: A basic tool of chemometrics. Chemometrics and Intelligent Laboratory Systems 58, 109-130 (2001).
89. S. de Jong, SIMPLS: An alternative approach to partial least squares regression. Chemometrics and Intelligent Laboratory Systems 18, 251-263 (1993).
90. K. Pearson, On lines and planes of closest fit to systems of points in space. The London, Edinburgh, and Dublin Philosophical Magazine and Journal of Science 2, 559-572 (1901).
91. C. Spearman, The proof of measurement of association between two things. The American Journal of Psychology 15, 72-101 (1904).
92. Y. Benjamini, Y. Hochberg, Controlling the false discovery rate: A practical and powerful approach to multiple testing. Journal of the Royal Statistical Society: Series B (Methodological) 57, 289-300 (1995).
93. V. D. Blondel, J. L. Guillaume, R. Lambiotte, R. Lefebvre, Fast unfolding of communities in large networks. Journal of Statistical Mechanics: Theory and Experiment 10, (2008).
94. S. Kim, ppcor: An R package for a fast calculation to semi-partial correlation coefficients. Communications for Statistical Applications and Methods 22, 665-674 (2015).
95. R. A. Fisher, On the interpretation of χ² from contingency tables, and the calculation of P. Journal of the Royal Statistical Society 85, 87-94 (1922).
96. T. M. Gihring, S. J. Green, C. W. Schadt, Massively parallel rRNA gene sequencing exacerbates the potential for biased community diversity comparisons due to variable library sizes. Environmental Microbiology 14, 285-290 (2012).
97. L. McInnes, J. Healy, N. Saul, L. Großberger, UMAP: Uniform Manifold Approximation and Projection. Journal of Open Source Software 3, (2018).
98. S. Brunton, J. Proctor, J. Kutz, Discovering governing equations from data by sparse identification of nonlinear dynamical systems. Proc Natl Acad Sci USA 113, 3932-3937 (2016).
99. B. de Silva et al., PySINDy: A Python package for the sparse identification of nonlinear dynamical systems from data. Journal of Open Source Software 5, (2020).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of decreasing the relative abundance of a member of the genus Streptococcus, a member of the genus Enterococcus, or a member of the genus Streptococcus and a member of the genus Enterococcus, in a patient that has undergone an intestinal surgery, the method comprising:
administering to the patient an effective amount of a composition comprising a fecal extract and a pharmaceutically acceptable carrier,
wherein the relative abundance of a member of the genus Streptococcus, a member of the genus Enterococcus, or a member of the genus Streptococcus and a member of the genus Enterococcus is reduced following administration of the composition.

2. A method of increasing the relative abundance of one or more members of the family Bacteroidaceae, one or more members of the family Lachnospiraceae, one or more members of the family Ruminococcaceae, one or more members of the family Akkermansiaceae, one or more members of the family Coriobacteriaceae, or any combination thereof in a patient that has undergone an intestinal surgery, the method comprising:
administering to the patient an effective amount of a composition comprising a fecal extract and a pharmaceutically acceptable carrier,
wherein the relative abundance of one or more members of the family Bacteroidaceae, the family Lachnospiraceae, the family Ruminococcaceae, the family Akkermansiaceae, or the family Coriobacteriaceae is increased following administration of the composition.

3. A method of increasing the presence of short-chain fatty acids (SCFAs), branched-chain fatty acids (BCFAs), or both SCFA and BCFAs in a patient that has undergone an intestinal surgery, the method comprising:
administering to the patient an effective amount of a composition comprising a fecal extract and a pharmaceutically acceptable carrier,
wherein the concentration of SCFAs, BCFAs, or both is increased following administration of the composition.

4. The method of claim 1, wherein the composition comprises a reconstituted freeze-dried composition comprising biological material, a cryoprotectant or lyoprotectant or both cryoprotectant and lyoprotectant,
wherein the freeze-dried composition is friable, and
wherein the biological material comprises human gut, colon, or intestinal fecal microbes.

5. The method of claim 4, wherein the lyoprotectant comprises trehalose.

6. The method of claim 1, wherein the composition further comprises a gut-transit protectant.

7. The method of claim 1, wherein the composition comprises no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% weight non-living material/weight biological material.

8. The method of claim 1, wherein the composition comprises 1×10¹⁰ to 5×10¹² cells.

9. The method of claim 1, wherein the relative abundance of Streptococcus, Enterococcus, or both Streptococcus and Enterococcus is reduced by at least 1%, at least 5%, or at least 10% following administration of the composition.

10. The method of claim 2, wherein the relative abundance of one or more members of the family Bacteroidaceae, one or more members of the family Lachnospiraceae, one or more members of the family Ruminococcaceae, one or more members of the family Akkermansiaceae, one or more members of the family Coriobacteriaceae, or any combination thereof, is increased by at least 1%, at least 5%, or at least 10% following administration of the composition.

11. The method of claim 10, wherein the one or more members of the family Bacteroidaceae, comprise one or more members of the genus Bacteroides, one or more members of the family Lachnospiraceae comprises one or more members of the genera Blautia, Coprococcus, Dorea, Lachnospira, Oribacterium, and Roseburia, one or more members of the family Ruminococcaceae comprises one or more members of the genera Ruminococcus and Faecalibacterium, one or more members of the family Akkermansiaceae comprises one or more members of the genus Akkermansia, and one or more members of the family Coriobacteriaceae comprises one or more members of the genus Collinsella.

12. The method of claim 3, wherein the concentration of SCFAs is increased by at least 1%, at least 5%, or at least 10% following administration of the composition.

13. The method of claim 1, wherein the composition is administered within 12 hours, within 24 hours, within 36 hours, within 48 hours, within 60 hours, or within 72 hours of the end of the intestinal surgery.

14. The method of claim 1, wherein the administering is by oral administration.

15. The method of claim 1, wherein the administering is by intubation.

16. The method of claim 15, wherein the intubation is by post-pyloric nasojejunal feeding tube.

17. The method of claim 9, wherein the relative abundance is determined 1 day, 3 days, 10 days, 14 days, 30 days, 90 days, or 180 days after the administration of fecal material.

18. The method of claim 3, wherein the concentration of one or more SCFA or BCFA is determined 1 day, 3 days, 10 days, 14 days, 30 days, 90 days, or 180 days after the administration of fecal material.
